SEQUENCE LISTING (50 sequences)

SEQ ID NO: 1 - protein, 13 aa, Homo sapiens
KGRRRRGAISAEV

SEQ ID NO: 2 - protein, 20 aa, Homo sapiens [sequence omitted]

SEQ ID NO: 3 - protein, 11 aa, Artificial
  synthetic polypeptide
ADKRRAVRIGA

SEQ ID NO: 4 - protein, 11 aa, Artificial
  synthetic polypeptide
ADKRRSVRIGA

SEQ ID NO: 5 - protein, 11 aa, Artificial
  synthetic polypeptide
RVVGGALRGAQ

SEQ ID NO: 6 - protein, 11 aa, Artificial
  synthetic polypeptide
RVVGGSLRGAQ

SEQ ID NO: 7 - protein, 11 aa, Artificial
  synthetic polypeptide
KLAGFAFKKNK

SEQ ID NO: 8 - protein, 11 aa, Artificial
  synthetic polypeptide
KLAGFSFKKNK

SEQ ID NO: 9 - protein, 11 aa, Artificial
  synthetic polypeptide
KFKRPALRRVR

SEQ ID NO: 10 - protein, 11 aa, Artificial
  synthetic polypeptide
KFKRPTLRRVR

SEQ ID NO: 11 - protein, 11 aa, Artificial
  synthetic polypeptide
FAFKKAFKLAG

SEQ ID NO: 12 - protein, 11 aa, Artificial
  synthetic polypeptide
FAFKKSFKLAG

SEQ ID NO: 13 - protein, 10 aa, Artificial
  synthetic polypeptide
ASQKRPAQRH

SEQ ID NO: 14 - protein, 10 aa, Artificial
  synthetic polypeptide
ASQKRPSQRH

SEQ ID NO: 15 - protein, 11 aa, Artificial
  synthetic polypeptide
LLRMFAFKAPA

SEQ ID NO: 16 - protein, 11 aa, Artificial
  synthetic polypeptide
LLRMFSFKAPA

SEQ ID NO: 17 - protein, 11 aa, Artificial
  synthetic polypeptide
IVRKAALRRLL

SEQ ID NO: 18 - protein, 11 aa, Artificial
  synthetic polypeptide
IVRKATLRRLL

SEQ ID NO: 19 - protein, 14 aa, Artificial
  synthetic polypeptide
ARKRERTYAFGHHA

SEQ ID NO: 20 - protein, 14 aa, Artificial
  synthetic polypeptide
ARKRERTYSFGHHA

SEQ ID NO: 21 - protein, 9 aa, Artificial
  synthetic polypeptide
RRRRAIIFI

SEQ ID NO: 22 - protein, 9 aa, Artificial
  synthetic polypeptide
RRRRSIIFI

SEQ ID NO: 23 - protein, 13 aa, Artificial
  synthetic polypeptide
FKLKRKGAFKKFA

SEQ ID NO: 24 - protein, 13 aa, Artificial
  synthetic polypeptide
FKLKRKGSFKKFA

SEQ ID NO: 25 - protein, 13 aa, Artificial
  synthetic polypeptide
RRFKRQGAFFYFF

SEQ ID NO: 26 - protein, 13 aa, Artificial
  synthetic polypeptide
RRFKRQGSFFYFF

SEQ ID NO: 27 - protein, 11 aa, Artificial
  synthetic polypeptide
KKKRFAFKKAF

SEQ ID NO: 28 - protein, 11 aa, Artificial
  synthetic polypeptide
KKKRFSFKKAF

SEQ ID NO: 29 - protein, 14 aa, Artificial
  synthetic polypeptide
NRFARKGALRQKNV

SEQ ID NO: 30 - protein, 14 aa, Artificial
  synthetic polypeptide
NRFARKGSLRQKNV

SEQ ID NO: 31 - protein, 14 aa, Artificial
  synthetic polypeptide
NRFARKGDLRQKNV

SEQ ID NO: 32 - protein, 73 aa, Sus scrofa
EEEEKKKQQEEEAERLRRIQEEMEKERKRREEDEERRRKEEEERRMKLEMEAKRKQEEEERKKREDDEKRKKK

SEQ ID NO: 33 - protein, 206 aa, Trichomonas vaginalis
EEEEKKKEEEEKKQKEEQERLAKEEAERKQKEEQERLAKEEAERKQKEEEERKQKEEEERKQKEEEERKLKEEQERKAAEEKKAKEEAERKAKEEQERKAEEERKKKEEEERLERERKEREEQEKKAKEEAERIAKLEAEKKAEEERKAKEEEERKAKEEEERKKKEEQERLAKEKEEAERKAAEEKKAKEEQERKEKEEAERKQR

SEQ ID NO: 34 - protein, 10 aa, Artificial
  synthetic polypeptide
LLRMFTKAPA

SEQ ID NO: 35 - protein, 13 aa, Artificial
  synthetic polypeptide
FKLKRKGTFKKFA

SEQ ID NO: 36 - protein, 14 aa, Artificial
  synthetic polypeptide
NRFARKGTLRQKNV

SEQ ID NO: 37 - protein, 51 aa, Homo sapiens
TLCGTPEYLAPEIILSKGYNKAVDWWALGVLIYEMAAGYPPFFADQPIQIY

SEQ ID NO: 38 - protein, 51 aa, Homo sapiens
TFCGTPEYLAPEVLEDNDYGRAVDWWGLGVVMYEMMCGRLPFYNQDHERLF

SEQ ID NO: 39 - protein, 59 aa, Homo sapiens
TFCGTPNYIAPEILRGDEYGFSVDWWALGVLMFEMMAGRSPFDIVGMQNSEENTEDYLF

SEQ ID NO: 40 - protein, 60 aa, Homo sapiens
TFCGTPNYIAPEILRGEDYGFSVDWWALGVLLYEMLAGRSPFDLAGASENPDQNTEDYLF

SEQ ID NO: 41 - protein, 58 aa, Homo sapiens
TFCGTPNYIAPEILRGEEYGFSVDWWALGVLMFEMMAGRSPFDIITDNPDMNTEDYLF

SEQ ID NO: 42 - protein, 60 aa, Homo sapiens
TFCGTPNYIAPEILRGEDYGFSVDWWALGVLMFEMMAGRSPFDIVGSSDNPDQNTEDYLF

SEQ ID NO: 43 - protein, 51 aa, Homo sapiens
TFCGTPDYIAPEILQGLKYSFSVDWWSFGVLLYEMLIGQSPFHGDDEDELF

SEQ ID NO: 44 - protein, 51 aa, Homo sapiens
TFCGTPDYIAPEILLGQKYNHSVDWWSFGVLVYEMLIGQSPFHGQDEEELF

SEQ ID NO: 45 - protein, 51 aa, Homo sapiens
TFCGTPDYIAPEIIAYQPYGKSVDWWSFGVLLYEMLAGQPPFDGEDEEELF

SEQ ID NO: 46 - protein, 51 aa, Homo sapiens
TFCGTPDYIAPEIIAYQPYGKSVDWWAYGVLLYEMLAGQPPFDGEDEDELF

SEQ ID NO: 47 - protein, 51 aa, Homo sapiens
TFCGTPDYIAPEIIAYQPYGKSVDWWAFGVLLYEMLAGQAPFEGEDEDELF

SEQ ID NO: 48 - protein, 51 aa, Homo sapiens; identical to SEQ ID NO: 47

SEQ ID NO: 49 - protein, 51 aa, Homo sapiens
TFCGTPDYIAPEILQELEYGPSVDWWALGVLMYEMMAGQPPFEADNEDDLF

SEQ ID NO: 50 - protein, 51 aa, Homo sapiens
TFCGTPDYIAPEILQEMLYGPAVDWWAMGVLLYEMLCGHAPFEAENEDDLF

(19) United States
(12) Patent Application Publication
(10) Pub. No.: US 2020/0181677 A1
(43) Pub. Date: Jun. 11, 2020

(54) SCREENING SYSTEM AND METHODS FOR IDENTIFYING ENZYME SUBSTRATES AND MODULATORS OF ENZYME ACTIVITY

(71) Applicant: Regents of the University of Minnesota, Minneapolis, MN (US)

(72) Inventors: Sivaraj Sivaramakrishnan, North Oaks, MN (US); Ruth Sommese, Groton, CT (US)

(21) Appl. No.: 16/619,798

(22) PCT Filed: Jun. 5, 2018
(86) PCT No.: PCT/US18/36148

§ 371 Date: Dec. 5, 2019

Related U.S. Application Data
(60) Provisional application No. 62/515,204, filed on Jun. 5, 2017.

(51) Int. Cl. C12Q 1/48; C12N 9/12; C07K 14/00; G01N 21/64

(52) U.S. Cl. CPC C12Q 1/485; C12N 9/1205; C07K 14/00; G01N 2500/02; G01N 21/6408; C07K 2319/50; C07K 2319/60; C12Y 207/11013

(57) ABSTRACT

Materials and methods for identifying modulators of protein function (e.g., enzyme activity), including modulators that target particular substrates for enzymes, are provided herein.

### CROSS-REFERENCE TO RELATED APPLICATIONS

This application claims benefit of priority from U.S. Provisional Application Ser. No. 62/515,204, filed on Jun. 5, 2017.

### STATEMENT AS TO FEDERALLY SPONSORED RESEARCH

This invention was made with government support under CA186752 and GM105646 awarded by the National Institutes of Health. The government has certain rights in the invention.

### TECHNICAL FIELD

This document relates to materials and methods that can be used to, for example, identify molecules that target particular substrates for enzymes, including materials and methods that can be used to identify molecules that target particular kinase substrates.

### BACKGROUND

The canonical role of protein kinases is to recognize, bind, and phosphorylate specific serine, threonine, or tyrosine residues on substrate proteins (Hornbeck et al., Nucleic Acids Res. 43:D512-520, 2015; and Pearce et al., Nature Rev. Mol. Cell. Biol. 11:9-22, 2010). Given that kinases are one of the largest gene families in eukaryotes and are involved in nearly every cellular function (Manning et al., Science 298:1912-1934, 2002), much work has focused on understanding the mechanisms that dictate substrate-kinase pairings (de Oliveira et al., Science Signaling 9:re3, 2016). The overlapping network of kinase-substrate interactions provides exquisite specificity in cell signaling pathways, but also presents challenges to understanding the mechanistic basis of biological processes.

After recognizing its substrate, the catalytic domain of a kinase transfers a phosphate group from ATP to a phospho-residue on the substrate. Phospho-transfer is followed by release of ADP and the phosphorylated substrate to prime the kinase for another catalytic cycle (Adams, Chem. Rev. 101:2271-2290, 2001). While the catalytic domains of eukaryotic kinases are structurally conserved (Hanks and Hunter, FASEB J 9:576-596, 1995; and Taylor and Komev Trends Biochem. Sci. 36:65-77, 2011), the local environment around the substrate-binding pocket of the kinase catalytic domain varies between kinases (Ubersax and Ferrell, Jr. Nature Rev. Mol. Cell. Biol. 8:530-541, 2007), leading to the view that phosphorylation site recognition may occur through conserved residues flanking the phospho-residue on the substrate.

### SUMMARY

This document is based, at least in part, on the development of materials and methods for using a library of sensors (e.g., molecules containing Förster resonance energy transfer [FRET] donors and acceptors or other reporter pairs that generate a quantifiable signal) to monitor transient kinase-substrate complexes. As described herein, quantitative FRET measurements were combined with traditional kinase activity assays to understand different catalytic states of the PKC-substrate interaction. In particular, a platform for pairwise comparison of different substrate peptides bound to the protein kinase C (PKC) catalytic domain, with or without various nucleotides or inhibitors, was used to elucidate several questions in substrate-kinase regulation: (1) how phosphorylation alters substrate binding affinity and kinase specific activity; (2) how substrate binding affinity correlates with kinase specific activity; (3) how substrate binding affinity alters catalytic activity in the presence of multiple substrates; and (4) how substrate binding affinity affects potency of substrate-competitive inhibitors. These studies revealed that preferential binding of non-phosphorylated versus phosphorylated substrates leads to enhanced kinase specific activity; that kinase specific activity is inversely correlated with substrate binding affinity; that high affinity substrates suppress phosphorylation of their low affinity counterparts; and that substrate-competitive inhibitors can displace low affinity substrates more potently than high affinity substrates, leading to substrate selective inhibition of kinase activity.

The systems and methods disclosed herein can be used to identify substrates and modulators (e.g., inhibitors and activators) for a variety of enzymes, including protein kinases (AGC-kinases as well as non-AGC kinases). These systems and methods, which can be based on detection of FRET and changes in FRET, provide novel approaches for exploring the impact of substrates and activity modulators on enzyme regulation.

In a first aspect, this document features a polypeptide containing, consisting essentially of, or consisting of a catalytic domain of an enzyme, a linker containing an amino acid sequence flanked by a resonance energy transfer donor and a resonance energy transfer acceptor (e.g., a FRET donor and a FRET acceptor), and a substrate having affinity for the catalytic domain of the enzyme, where the linker separates the catalytic domain from the substrate. The enzyme can be a kinase (e.g., a member of the AGC class of kinases. For example, the kinase can be a protein kinase C (e.g., PKCα), or a protein kinase A (PKA). The linker can include an ER/K sequence containing a substantially repeating sequence of glutamic acid, arginine, and lysine residues, where the ER/K sequence is about 50 to about 250 amino acids in length. The linker can be about 10 nm to about 30 nm in length. The linker can contain the amino acid sequence set forth in SEQ ID NO:32 or a sequence that is at least 95% identical to the sequence set forth in SEQ ID NO:32. The linker can further contain a protease cleavage site (e.g., an N-terminal tobacco etch virus (TEV) protease cleavage site) between the ER/K sequence and the FRET donor or acceptor that is closest to the peptide sequence. The polypeptide can further contain a (Gly-Ser-Gly)₂₋₄ amino acid sequence on either side of the FRET acceptor and the FRET donor, and between the protease cleavage site and the ER/K sequence. The substrate can be a peptide. The peptide can be 5 to 20 amino acids in length. In some cases, the enzyme can be PCKα, and the substrate can be a peptide that is 5 to 20 amino acids in length. The peptide may contain, for example, the sequence set forth in SEQ ID NO:29.

In another aspect, this document features a polypeptide containing, consisting essentially of, or consisting of, in order from amino terminus to carboxy terminus, a molecule having affinity for a catalytic domain of an enzyme, a first (Gly-Ser-Gly)₂₋₄ amino acid sequence, a resonance energy transfer donor or acceptor, a second (Gly-Ser-Gly)₂₋₄ amino acid sequence, a protease cleavage site (e.g., a TEV protease cleavage site), a third (Gly-Ser-Gly)₂₋₄ amino acid sequence, a linker containing the amino acid sequence set forth in SEQ ID NO:32 or a sequence at least 95% identical to the sequence set forth in SEQ ID NO:32, a fourth (Gly-Ser-Gly)₂₋₄ amino acid sequence, a partner for the resonance energy transfer donor or acceptor, a fifth (Gly-Ser-Gly)₂₋₄ amino acid sequence, and the catalytic domain of the enzyme or a portion of the catalytic domain for which the molecule has affinity. The number of (Gly-Ser-Gly) repeats in the first, second, third, fourth, and fifth (Gly-Ser-Gly)₂₋₄ sequences can independently be two, three, or four. The enzyme can be PCKα, and the molecule having affinity for the catalytic domain can be a peptide having a length of 5 to 20 amino acids. In some cases, the peptide can contain the sequence set forth in SEQ ID NO:29.

In another aspect, this document features a nucleic acid encoding a polypeptide as disclosed herein. For example, this document features a polypeptide containing a catalytic domain of an enzyme, a linker containing an amino acid sequence flanked by a resonance energy transfer donor and a resonance energy transfer acceptor (e.g., a FRET donor and a FRET acceptor), and a substrate having affinity for the catalytic domain of the enzyme, where the linker separates the catalytic domain from the substrate. In addition, this document features a host cell containing the nucleic acid.

In another aspect, this document features a method for identifying a modulator of enzyme activity, where the method includes (a) providing a polypeptide sensor, the sensor containing (i) a catalytic domain of an enzyme, (ii) a linker containing an amino acid sequence flanked by a FRET donor and a FRET acceptor, and (iii) a substrate for the enzyme, where the linker separates the catalytic domain from the substrate; (b) measuring an initial FRET ratio for the sensor; (c) contacting the sensor with a candidate molecule; (d) measuring a test FRET ratio for the sensor; and (e) identifying the candidate molecule as a modulator of the enzyme activity when the test FRET ratio is increased or decreased by at least 10% as compared to the initial FRET ratio. The enzyme can be a kinase (e.g., a member of the AGC class of kinases. For example, the kinase can be a PKC (e.g., PKCα), or a PKA. The linker can include an ER/K sequence containing a substantially repeating sequence of glutamic acid, arginine, and lysine residues, where the ER/K sequence is about 50 to about 250 amino acids in length. The linker can be about 10 nm to about 30 nm in length. The linker can contain the amino acid sequence set forth in SEQ ID NO:32 or a sequence that is at least 95% identical to the sequence set forth in SEQ ID NO:32. The linker can further contain a protease cleavage site (e.g., a TEV protease cleavage site) between the ER/K sequence and the FRET donor or acceptor that is closest to the peptide sequence. The polypeptide can further contain a (Gly-Ser-Gly)₂₋₄ amino acid sequence on either side of the FRET acceptor and the FRET donor, and between the protease cleavage site and the ER/K sequence. The substrate can be a peptide. The peptide can be 5 to 20 amino acids in length. In some cases, the enzyme can be PCKα, and the substrate can be a peptide that is 5 to 20 amino acids in length. The peptide may contain, for example, the sequence set forth in SEQ ID NO:29. The method can further include providing two or more sensors, each containing a different substrate for the enzyme, measuring initial and test FRET ratios for each substrate, and identifying the candidate compound as a substrate-specific modulator of the enzyme activity when at least some of the test FRET ratios are not increased or decreased by at least 10% as compared to the initial FRET ratios.

In still another aspect, this document features a method for identifying a modulator of enzyme activity, where the method includes (a) providing a polypeptide sensor, the sensor containing (i) a catalytic domain of an enzyme, (ii) a linker containing an amino acid sequence flanked by a FRET donor and a FRET acceptor, and (iii) a candidate modulator for the enzyme, where the linker separates the catalytic domain from the candidate modulator; (b) measuring an initial FRET ratio for the sensor; (c) contacting the sensor with a substrate for the enzyme; (d) measuring a test FRET ratio for the sensor; and (e) identifying the candidate as a modulator of the enzyme when the test FRET ratio is within about 10% of the initial FRET ratio. The method can further include contacting different preparations of the sensor with different substrates for the enzyme, measuring initial and test FRET ratios for each substrate, and identifying the candidate modulator as a substrate-specific modulator of the enzyme when at least some of the test FRET ratios are increased or decreased by at least 10% as compared to the initial FRET ratios.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representative schematic of a PKCα peptide FRET sensor as described herein. The PKCα peptide sensor utilizes an ER/K FRET linker to separate the catalytic domain of PKCα from a variety of short peptides ≤14 amino acid residues in length (FIG. 2). In the majority of the experiments described in the Examples section herein, the fluorophores used with the ER/K linker were monomeric Cerulean (mCer, FRET donor) and monomeric Citrine (mCit, FRET acceptor). For the time-resolved measurements shown in FIG. 4A, however, monomeric eGFP and monomeric Cherry were used.

FIG. 2 is a table showing the amino acid sequences of PKCα FRET sensors (peptides 1-14; SEQ ID NOS:3-31). Peptides used in activity assays were Ser- or Thr-containing, or FRET-based sensors (Ala- or Asp-containing). In the amino acid sequences, the phosphorylation site (Ser or Thr) is italicized. Underlined (hydrophobic) or bold (basic) text indicates consensus site residues important for phosphorylation. The basal FRET ratio is shown for each alanine-containing sensor, and was derived from at least three protein preparations (N≥3 preparations).

FIGS. 3A-3F demonstrate that the loss of affinity for phosphorylated substrate enhances kinase specific activity. FIGS. 3A to 3C are a series of graphs showing an increase in the FRET ratio (ΔFRET) upon addition of 100 μM ATP, 100 μM ADP, or 100 μM Sangivamycin (Sang) for sensors containing short alanine-substituted substrate peptides and either the catalytic domain of PKCα (FIGS. 3A and 3C) or the catalytic domain of PKA (FIG. 3B). Peptide sequence information is provided in FIG. 2. FIG. 3D is a graph plotting the FRET ratio for PKCα peptide #14 sensors containing either an alanine (S14A), serine (S14S), or aspartic acid (S14D, phosphomimetic) with 100 μM ATP FIG. 3E is a schematic showing a subset of the steps in the catalytic cycle of PKCα. The gray scale shading denotes the steady-state FRET ratios of the PKCα peptide #14 sensors containing either an aspartic acid (S14D) or an alanine residue (S14A) with 100 μM ATP or 100 μM ADP States are arranged based on progression of FRET through the catalytic cycle, and do not indicate the relative prevalence of all possible states. FIG. 3F is a graph plotting ATP consumption of the catalytic domain of PKCα for serine-containing peptide #14 upon addition of equal concentrations of either aspartic acid-substituted (S14D) or alanine-substituted (S14A) peptide. For all FRET and activity readings, data were derived from at least three independent protein preparations, with at least two replicated measurements for each condition per experiment (mean±SEM, N≥3 experiments).

FIGS. 4A and 4B demonstrate the inverse correlation between substrate affinity and kinase activity. FIG. 4A is a graph plotting a comparison between steady-state FRET and fractional binding for four alanine-containing peptide sensors. For fractional binding, fluorescence decay single photon counting data was fit to I(t)=A₁e^{-t/τ}^{¹}+A₂e^{-t/τ}^{²}, where τ₁ was set to the lifetime of a 10 nm ER/K linker control (see, TABLE 1). The mol fraction of the bound state(s) was calculated as A₂/(A₁+A₂).

FIG. 4B is a graph plotting the steady-state FRET of PKCα catalytic domain sensors with fourteen short peptides (FIG. 2; SEQ ID NOS:4, 6, 8, 10, 12, 14, 16, 18, 20, 22, 24, 26, 28, and 30) compared to activity of the catalytic domain with corresponding substrate peptides. Activity assays were performed at 21-22° C. with equimolar peptide concentrations (500 μM) and kinase (50 nM). Specific activity is reported as mol ATP consumed per mol kinase per second (s⁻¹). In all experiments, data were derived from at least three protein preparations (mean±SEM, N≥3 experiments).

FIGS. 5A and 5B show that high affinity substrates suppress phosphorylation of low affinity counterparts. FIG. 5A includes schematics and a graph the relative activity of sensors containing either the alanine-substituted peptide #14 with different linker distances (10 nm vs. 30 nm; 1 vs 2) or containing different alanine-substituted peptides (peptide #8 vs #11; 3 vs 4; black) with the same 10 nm linker. Activity data is plotted versus the steady-state FRET ratio of the peptide sensors. FIG. 5B is a graph plotting the activity of the isolated catalytic domain for peptide #6, peptide #12, and equimolar concentrations of peptides #6 and #12. For both FRET and activity experiments, data are derived from at least three protein preparations with at least two replicates for each condition per independent experiment (mean±SEM, N≥3 experiments).

FIGS. 6A-6D demonstrate that kinase-substrate binding affinity impacts inhibitor potency. FIG. 6A is a graph plotting titration of peptide #8 and peptide #11 FRET sensors in the presence of 100 μM ATP by the substrate competitive inhibitor Bisindolylmaleimide I (BimI). The FRET ratio for each sensor was normalized to the FRET ratio of that sensor in the absence of any inhibitor. FIG. 6B is a graph plotting the inhibition of PKCα catalytic domain activity by 0.1 μM BimI for three different substrate peptides (order of peptide affinity: 11>8>6). Activity assays were performed with equimolar peptide concentrations (500 μM) and kinase (50 nM), and specific activity is reported as mol ATP consumed per mol kinase per second (s⁻¹). FIG. 6C is a graph plotting the percent inhibition of PKCα catalytic domain activity by 0.1 μM BimI for substrate peptides in FIG. 6B. Percent inhibition is plotting versus the steady-state FRET ratio for the corresponding alanine-substituted FRET sensors. For all FRET and activity readings, data were derived from at least three protein preparations with at least two replicates per independent experiment (mean±SEM, N≥3 experiments). FIG. 6D is a schematic showing that for PKCα, varying substrate affinity reduces the potency of substrate-competitive inhibitors such as BimI.

FIG. 7 is a graph plotting the results of a drug screen using different types of kinase inhibitors on the interaction between the PKCα catalytic domain and the EGFR substrate peptide (FIG. 2, SEQ ID NO:17) as quantified by the change in FRET ratio (ΔFRET) between untreated and inhibitor treated conditions.

FIGS. 8A-8D are parts of a schematic illustrating the combined use of computational and experimental approaches to dissect kinase-substrate interaction. In FIG. 8A, PKC-substrate interaction is homology modeled from the PKCι-Par3 interaction (PDB ID 5HI1). In FIG. 8B, generalized Newton-Euler inverse mass operator (GNEIMO) torsional molecular dynamics (MD) is used to obtain a kinase-substrate conformational ensemble. In FIG. 8C, structural analysis is used to identify hot spot residues and motifs within the kinase and substrate that contribute to binding and catalytic activity. In FIG. 8D, SPASM FRET sensors are used to test GNEIMO-MD predictions using site-directed mutagenesis. Experimental results then can be used to validate GNEIMO-MD predictions and refine computational methods.

FIGS. 9A-9E show that electrostatic interactions between residues in the N terminus of the peptide substrate and the kinase catalytic domain are major determinants of binding strength. FIG. 9A shows the predicted binding site of the tight binding peptide substrate p12 in PKCα. The backbone atoms of the residues before the Ser that is phosphorylated in the substrate p12 are shown in dark gray, and the residues after the Ser are shown in white. The residues in the peptide substrate are shown in sticks. The residues in PKCα that make strong contact with the peptide are labeled and shown in ball and sticks; the numbering is standardized such that the phosphorylated Ser/Thr is numbered as position 9. FIG. 9B is a graph plotting the calculated average interaction energy of the N terminus of the peptide substrates with PKCα versus FRET intensity ratio. Weak binding peptide substrates (1-7) are at the left portion of the graph, medium binding peptide substrates (8 and 9) are in the middle portion of the graph, and strong binding peptide substrates (10-14) are in the right portion of the graph. The error bars for FRET measurement and the non-bond interaction energy represent the mean±S.E. and the standard deviation, respectively. FIG. 9C is a graph showing that the FRET ratio of SPASM sensors have a linear correlation with a recognition metric derived by Nishikawa et al. (J. Biol. Chem. 272:952-960, 1997). FIG. 9D is a series of graphs showing the effect on binding affinity of site-directed mutagenesis of residues in peptides p1, p2, p4, and p6 identified from the MD simulation analysis. Mutagenesis of N-terminal residues to Arg resulted in enhanced kinase-substrate interaction, as measured by the FRET ratio. FIG. 9E is a graph plotting the kinase activity of the mutant peptide substrates shown in FIG. 9D. Mutagenesis of N-terminal residues to Arg marginally increased kinase-specific activity, as compared with the corresponding wild-type peptides. The FRET results are expressed as mean±S.E. of three independent experiments performed in triplicate (n≥9). *, p<0.05; **, p<0.005; ***, p<0.0005; ****, p<0.0001.

FIGS. 10A-10F show that high activity peptides populate catalytically favorable conformations when bound to the kinase. FIGS. 10A and 10B are snapshots from MD trajectories showing the plausible catalytic conformation of the high activity peptide p2 (FIG. 10A) and low activity peptide p12 (FIG. 10B), bound to PKCα. These snapshots were selected based on the shortest distance between the Ser in the substrate to Asp⁴⁶⁶ that abstracts the proton from Ser and also to the γ-PO₄ group of the ATP. FIG. 10C is a pair of graphs showing the population distribution of various peptide conformations from three 100-ns all-atom MD trajectories for peptide substrates p2 and p12. The MD ensemble was clustered by two distances—the distance between ATP(γP) and S/T(OG) of the substrate peptide and between D466(OD) and S/T(OG). The cross marks in these two figures are the conformations for which the distances are shown in FIGS. 10A and 10B. FIG. 10D is a graph plotting the population density of the catalytic conformations as captured in the MD simulations. Cutoffs of 4.2 Å in the ATP(γP) and S/T(OG) distance and 4.1 Å in the D466(OD) and S/T(OG) distance were used to calculate the population density. FIG. 10E is a snapshot from MD trajectories showing the rotamer flip change in the χ₁ angle of the pSer/Thr in peptide substrates as seen from crystal structures before (PDB code 4XW5, PKA) and after (PDB code 4IAK, PKA) phosphoryl transfer. FIG. 10F is a snapshot showing all-atom MD simulations after the PO₄ transfer, with a rotamer flip in pSer in the “good activity” peptide substrate p1 after the PO₄ transfer to the pSer in the peptide. The panel shows the most populated rotamer of Ser/Thr in the simulations of the peptide p1 before and after the PO₄ transfer. In FIGS. 10E and 10F, the Ser and pSer are shown in sticks.

FIGS. 11A-11D show that an Arg positioned two or three residues C-terminal to the phosphorylated serine in the peptide substrate is necessary and sufficient for high kinase activity. FIG. 11A is a pair of graphs plotting the time variation of the distance between the γ-PO₄ group of ATP and the OG atom in Ser, and the distance between residue at position 11 to the γ-PO₄ group of ATP, in peptide substrates p2 and p12. FIG. 11B is a representative snapshot from the MD simulations, showing the close distance between the side chains of Arg11 and Ser9 of peptide p2 to the γ-PO₄ group of ATP. FIG. 11C is a series of graphs plotting the kinase activity measured for mutants at positions 11 and 12 of peptide substrates p9, p10, p12, and p2, indicating that Arg at position 11 or 12 is necessary for high kinase activity. FIG. 11D is a series of graphs plotting the FRET ratio of peptide substrates. Results are expressed as mean±S.E. of three independent experiments performed in triplicate (n≥9). *, p<0.05; **, p<0.005; ***, p<0.0005; ****, p<0.0001.

FIG. 12 is a sequence alignment of segments of PKA and PKC family members. Residues lining the binding groove of the N-terminus of the peptide substrates are marked with arrows. These residues are either conserved or conservative replacements among the PKC isozymes.

FIG. 13 is a series of graphs plotting the population density of the root-mean-squared fluctuations (RMSF) of heavy atoms, showing the level of flexibility for every residue in peptides p10 and p13. The number at the upper left of each graph represents the relative positions of the residue in the peptide substrate, relative to the p-Ser/Thr. The residues C-terminal of p-Ser9 in p13 (residues 10, 11, 12, and 13) showed higher flexibility than those in p10.

FIG. 14 is a diagram illustrating distinct mechanisms of kinase activity and substrate-binding strengths. The left panel illustrates that high activity peptides display greater conformational flexibility in the domain C-terminal to the phosphorylated Ser/Thr (C-domain). An Arg/Lys residue 2 to 3 amino acids C-terminal to the phosphorylated Ser/Thr threads the formation of a catalytically active conformation. The right panel illustrates that tight binding peptides have several Arg/Lys residues N-terminal to the phosphorylated Ser/Thr that undergo strong columbic interactions with an acidic patch in the kinase catalytic domain.

### DETAILED DESCRIPTION

The materials and methods described herein provide means for assessing and monitoring transient interactions between molecules, such as enzymes and substrates, or other proteins having a function that can be modulated by interaction with an activator or inhibitor, for example. Thus, this document provides, inter alia, chimeric polypeptides that can be used as a tool to determine whether a candidate compound (e.g., a peptide, nucleic acid, small molecule, or biologic) is a substrate for a particular enzyme, or to determine whether a candidate compound is a modulator of enzyme activity. The materials and methods provided herein can be used, in some cases, to identify molecules (e.g., enzyme substrates) that can bind to selected polypeptides, and to identify and evaluate modulators (e.g., inhibitors or activators) of protein function (e.g., the function of enzymes such as, without limitation, kinases).

As described herein, this document provides polypeptides, nucleic acids encoding the polypeptides, host cells containing the nucleic acids and/or polypeptides, and methods for using the polypeptides and nucleic acids.

The term “polypeptide” as used herein refers to a compound of two or more subunit amino acids, regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers.

By “isolated” or “purified” with respect to a polypeptide it is meant that the polypeptide is separated to some extent from cellular components with which it normally is found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

The terms “nucleic acid” and “polynucleotide” can be used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

The nucleic acids may be incorporated into or contained within recombinant nucleic acid constructs such as vectors. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more “expression control sequences” that control or regulate the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

Host cells containing a nucleic acid or vector also are provided herein. Suitable host cells can include, without limitation, bacterial cells, insect cells (e.g., Sf9 cells), yeast cells, and human or non-human mammalian cells (e.g., HEK 293 cells, 3T3 cells, or HeLa cells). In addition to host cells, cell-derived extracts and cell-free systems, regardless of source, also can be used.

In certain embodiments, this document provides chimeric sensor polypeptides that contain (a) a catalytic domain of an enzyme, or a portion of a catalytic domain, where the portion includes the substrate-binding region; (b) a linker containing an amino acid sequence flanked by a FRET donor and a FRET acceptor; and (c) a substrate having affinity for the catalytic domain of the enzyme, or a candidate substrate that may have affinity for the catalytic domain of the enzyme. Interactions between the substrate or candidate substrate and the catalytic domain or portion thereof, or a change in the interaction between the substrate/candidate substrate and the catalytic domain or portion thereof, can be detected based on a FRET ratio measured for the sensor, or based on a change in the FRET ratio for the sensor before and after addition of a modulator of the enzyme's activity, for example. The polypeptides described herein can be employed in live cells or in cell extracts or lysates, or can be isolated from live cells, for the purpose of, for example, monitoring interactions within the polypeptides or between the polypeptides and other molecules.

In some cases, the FRET donor and FRET acceptor can be replaced by split-enzymes such as split-GFP, split-luciferase, or split-enzymes (distinct from the catalytic domain of the enzyme in (a) above) that show an increase in fluorescence, luminescence, or enzyme activity in response to an interaction between the catalytic domain of the enzyme (a) and the substrate (c). In some cases, the substrate can be replaced by a regulatory protein or polypeptide that may influence the enzymatic activity of the catalytic domain. Further, in some cases, FRET between the donor and the acceptor can be detected using metrics beyond the FRET ratio, including but not limited to FRET efficiency and lifetime of donor fluorescence.

The catalytic domain or portion thereof can be from any suitable enzyme, including, for example, an oxidoreductase, a transferase, a hydrolase, a lyase, an isomerase, or a ligase. In some embodiments, the catalytic domain or portion thereof can be from a kinase (e.g., a member of the AGC class of kinases, such as a protein kinase A (PKA), a protein kinase C (PKC), a protein kinase G (PKG), Akt, or S6K, or a kinase that is not a member of the AGC class, such as a CMGC type kinase, a calmodulin/calcium regulated kinase, or a tyrosine kinase). Amino acid sequences for such enzymes include those known in the art and available in, for example, GENBANK®. In some cases, a chimeric polypeptide can include an amino acid sequence that is at least 90% (e.g., at least 91%, at least 92%, at least 93%, at least 94%, at least 95%, at least 96%, at least 97%, at least 98%, or at least 99%) identical to a known amino acid sequence for an enzyme catalytic domain. It is to be noted that the enzyme need not be catalytically active. For example, one or more amino acid residues in the enzyme can be changed (mutagenized) to render the enzyme inactive, or the native enzyme may lack catalytic activity (e.g., can be a pseudoenzyme).

The linker can be positioned between the enzyme catalytic domain and the substrate or candidate substrate. Such positioning can permit the catalytic domain and the candidate substrate to move relative to one another. The linker can have a length of about 8 nm to about 35 nm (e.g., about 8 to 10 nm, about 10 to 15 nm, about 15 to 20 nm, about 20 to 25 nm, about 25 to 30 nm, about 30 to 35 nm, or about 10 to 30 nm). In some embodiments, the polypeptide linker can include an “ER/K” sequence that includes a substantially repeating sequence of glutamic acid, arginine, and lysine residues. For example, the linker may include the sequence set forth in SEQ ID NO:32 (EEEEKKKQQ EEEAERLRRIQEEMEKERKRREEDEERRRKEEEERRMKLEMEAKRKQEEEER KKREDDEKRKKK), or a sequence that is at least 90% (e.g., at least 91%, at least 92%, at least 93%, at least 94%, at least 95%, at least 96%, at least 97%, at least 98%, at least 99%, 90 to 93%, 93 to 95%, 95 to 98%, or 98 to 99%) identical to SEQ ID NO:32, or the linker may include the sequence set forth in SEQ ID NO:33 (EEEEKKKEEEEKKQKEE QERLAKEEAERKQKEEQERLAKEEAERKQKEEEERKQKEEEERKQKEEEERK LKEEQERKAAEEKKAKEEAERKAKEEQERKAEEERKKKEEEERLERERKERE EQEKKAKEEAERIAKLEAEKKAEEERKAKEEEERKAKEEEERKKKEEQERLA KEKEEAERKAAEEKKAKEEQERKEKEEAERKQR), or a sequence that is at least 90% (e.g., at least 91%, at least 92%, at least 93%, at least 94%, at least 95%, at least 96%, at least 97%, at least 98%, at least 99%, 90 to 93%, 93 to 95%, 95 to 98%, or 98 to 99%) identical to SEQ ID NO:33. Typically, the linker can be about 50 to about 250 (e.g., about 50 to about 75, about 75 to about 100, about 50 to about 100, about 100 to about 150, about 150 to about 200, about 100 to about 200, or about 200 to about 250) amino acids in length.

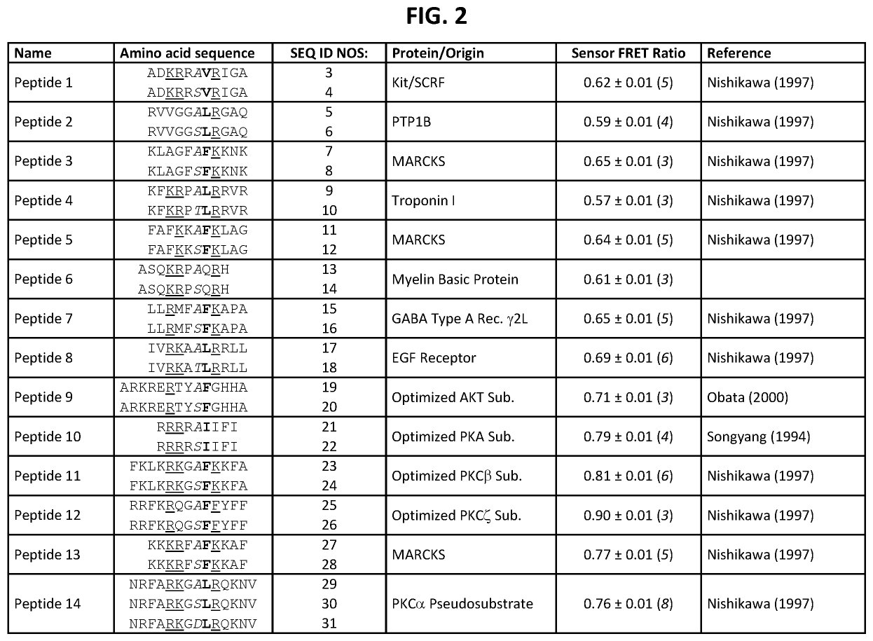

The substrate portion of the chimeric polypeptide can be, for example, a peptide having a length of about five amino acids to about 25 amino acids (e.g., 5 to 8, 5 to 10, 8 to 12, 10 to 15, 15 to 20, 18 to 20, or 20 to 25 amino acids). Particular examples of candidate substrate peptides that may bind to one or more kinase catalytic domains, and may be useful in the methods described herein, are shown in SEQ ID NOS:3 to 31 and 34 to 36 (FIG. 2 and TABLE 2), but it is noted that these sequences are not intended to be limiting. Further, the substrate portion of the chimeric polypeptides provided herein may include other types of molecules.

In some cases, the linker segment of a chimeric polypeptide can further include a protease cleavage site (e.g., an N-terminal tobacco etch virus (TEV) protease cleavage site, a Rhinovirus 3C protease cleavage site, or a thrombin cleavage site) between the ER/K sequence and the FRET donor or acceptor that is closest to the peptide sequence. The presence of a TEV protease cleavage site can allow for separation of the catalytic sequence and the substrate or candidate substrate, such that they are not covalently linked in the same polypeptide after protease cleavage takes place.

In addition, one or more (e.g., two, three, four, or more than four) (Gly-Ser-Gly)₂₋₄ sequences can be present on either side of the FRET acceptor and the FRET donor, and/or between the TEV protease cleavage site and the ER/K sequence. The inclusion of one or more Gly-Ser-Gly sequences can provide rotational freedom between the catalytic domain and the candidate substrate, which may facilitate interaction between those regions of the chimeric polypeptide.

Thus, in some cases, the chimeric polypeptides provided herein can include a catalytic domain of an enzyme or a portion thereof, a linker amino acid sequence that contains a sequence flanked by a FRET donor and a FRET acceptor, and a candidate substrate, in addition to one or more of optional components (e.g., a TEV protease cleavage site and/or one or more (Gly-Ser-Gly)₂₋₄ sequences. For example, in some embodiments a chimeric polypeptide may contain, in order from amino terminus to carboxy terminus, a candidate substrate having affinity for the catalytic domain of an enzyme, a (Gly-Ser-Gly)₂₋₄ amino acid sequence, a resonance energy transfer donor or acceptor, a (Gly-Ser-Gly)₂₋₄ amino acid sequence, a TEV protease cleavage site, a (Gly-Ser-Gly)₂₋₄ amino acid sequence, a linker containing an amino acid sequence at least 90% identical to the sequence set forth in SEQ ID NO:32 or SEQ ID NO:33, a (Gly-Ser-Gly)₂₋₄ amino acid sequence, a partner for the resonance energy transfer donor or acceptor, a (Gly-Ser-Gly)₂₋₄ amino acid sequence, and the catalytic domain of the enzyme, or a portion of the catalytic domain for which the candidate substrate as affinity.

This document also provides nucleic acids that encode the chimeric polypeptides described herein, as well as host cells containing the nucleic acids.

Also provided herein are methods that can be used to identify modulators of protein function (e.g., methods for identifying substrate-specific modulators of enzyme activity), methods for identifying substrates having specific affinity for one or more particular enzymes or catalytic domains, and methods for identifying enzymes or catalytic domains that have selective activity for one or more particular substrates. A modulator of enzyme activity can be, for example, an orthostatic modulator that interacts with the active site of the enzyme, an allosteric modulator that does not bind directly to the active site, or a bi-topic modulator that interacts with both the active site and with an allosteric site of the enzyme.

In some embodiments, for example, this document provides methods for identifying compounds that can modulate the activity of an enzyme. The methods can include providing a chimeric polypeptide sensor as disclosed above, for example, where the chimeric polypeptide sensor includes (1) the catalytic domain of the enzyme, or a portion of the catalytic domain that contains the region recognized by substrates for the enzyme; (2) a linker that includes an amino acid sequence flanked by a FRET donor and a FRET acceptor; and (3) a substrate for the enzyme, such that the linker separates the catalytic domain from the substrate. The methods also can include measuring an initial FRET ratio for the sensor, contacting the chimeric polypeptide sensor with a candidate compound, measuring a second FRET ratio for the sensor, and identifying the candidate compound as a modulator of the enzyme's activity if the second FRET ratio is increased or decreased by at least 10% (e.g., at least 10%, at least 25%, at least 50%, at least 75%, at least 100%, or more than 1000%) as compared to the initial FRET ratio.

This document also provides methods for identifying modulators of enzyme activity that can include providing a polypeptide sensor containing (1) a catalytic domain of the enzyme, or a portion of the catalytic domain that contains the region recognized by substrates for the enzyme; (2) a linker containing an amino acid sequence flanked by a FRET donor and a FRET acceptor; and (3) a candidate modulator for the enzyme, where the linker is positioned between the catalytic domain (or portion thereof) and the candidate modulator. The method can further include measuring an initial FRET ratio for the sensor, contacting the sensor with a substrate for the enzyme, measuring a second FRET ratio for the sensor, and identifying the candidate as a modulator of the enzyme when the test FRET ratio is within about 10% of the initial FRET ratio. The method also may be used to identify substrate-specific modulators of the enzyme's activity. For example, different preparations of the sensor can be contacted with different substrates for the enzyme, and an initial FRET ratio and a second FRET ratio can be determined for each substrate. The candidate modulator within the sensor can be identified as a substrate-specific modulator of the enzyme's activity when at least some of the test FRET ratios are increased or decreased by at least 10% as compared to the initial FRET ratios.

In some cases, this method can be used to identify substrate-specific modulators of the enzyme's activity. For example, a method above can be conducted using a plurality of chimeric sensor polypeptides (e.g., two, three, four, five, or more than five chimeric sensor polypeptides), where each sensor contains a different substrate for the enzyme. In such methods, an initial FRET ratio can be determined for each sensor, the sensors can be contacted with a candidate modulator compound, and a second FRET ratio can be determined for each sensor. If some sensors exhibit FRET ratios that are increased or decreased by at least 10% after addition of the candidate compound (as determined by comparing the second FRET ratio to the initial FRET ratio), but other sensors are do not exhibit FRET ratios that are increased or decreased by at least 10% after addition of the candidate compound, the candidate compound can be identified as a substrate-specific modulator of the enzyme's activity.

In some cases, the methods provided herein can be used to identify substrates having higher affinity for a particular enzyme. For example, a method can be conducted using a plurality of chimeric sensor polypeptides (e.g., two, three, four, five, or more than five chimeric sensor polypeptides), where each sensor contains a different substrate sequence, and where each sensor also includes a catalytic domain or a portion of a catalytic domain that includes the substrate binding region, separated from the substrate portion of the sensor by a FRET donor-linker-FRET acceptor as described herein. In such methods, a FRET ratio can be determined for each sensor. Sensors exhibiting higher FRET ratios can be identified as containing a substrate with higher affinity for the tested catalytic domain or portion thereof.

In some cases, the methods provided herein can be used to identify enzymes that display selective activity for a particular substrate. For example, a method can be conducted using a plurality of chimeric sensor polypeptides (e.g., two, three, four, five, or more than five chimeric sensor polypeptides), where each sensor contains a different enzyme (or a portion of an enzyme containing a catalytic domain or a section of a catalytic domain that includes the substrate binding region) that may act on a particular substrate, and where each sensor also includes the substrate of interest, separated from the enzyme portion of the sensor by a FRET donor-linker-FRET acceptor as described herein. In such methods, a FRET ratio can be determined for each sensor. Sensors exhibiting higher FRET ratios can be identified as containing an enzyme (or a portion of an enzyme) having selective activity for the tested substrate. In some cases, an initial FRET ratio and a second FRET ratio can be determined for each sensor before and after addition of a candidate modulator compound. If some sensors exhibit FRET ratios that are increased or decreased by at least 10% after addition of the candidate compound (as determined by comparing the second FRET ratio to the initial FRET ratio), but other sensors are not associated with FRET ratios that are increased or decreased by at least 10%, the candidate compound can be identified as an enzyme-specific modulator of the substrate's activity.

The invention will be further described in the following examples, which do not limit the scope of the invention described in the claims.

### EXAMPLES

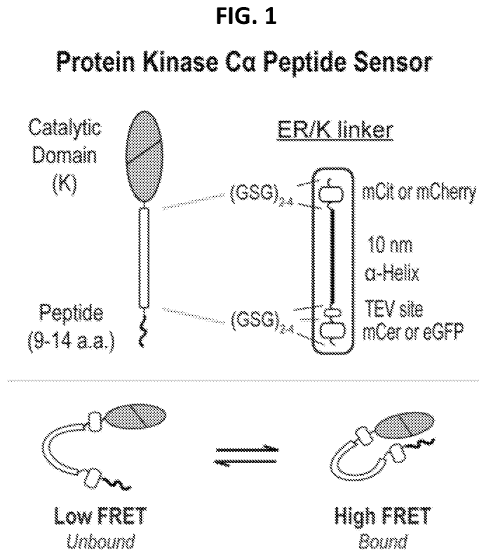

The studies described herein utilized PKCα peptide sensors based around an ER/K linker, which separates the catalytic domain of PKCα and a variety of short peptides ≤14 residues in length (FIG. 1). These peptides were based off the phosphorylation sites of known PKC substrates or predicted optimal PKC substrate peptides (Nishikawa et al., supra). To prevent phosphorylation, an alanine residue was substituted for the serine or threonine phospho-residue (FIG. 2). The linker itself contained an ER/K α-helix, ˜10 nm in length, having the amino acid sequence EEEEKKKQQEEEAERLRRIQEEMEKERKRREEDEERRRKEEEERRMKLEMEA KRKQEEEERKKREDDEKRKKK (SEQ ID NO:32) flanked by a FRET donor (mCerulean or GFP) and a FRET acceptor (mCitrine or mCherry, respectively) (Sivaramakrishnan and Spudich (2011) Proc. Natl. Acad. Sci. USA 108:20467-20472). The ER/K FRET linker also contained an N-terminal TEV protease cleavage site, and each discrete unit of this ER/K linker was separated by (Gly-Ser-Gly)₂₋₄ linkers (GSG) to provide rotational freedom. FIG. 2 lists the sequences along with the basal FRET ratios for each peptide sensor.

### Example 1—Experimental Procedures: Assessing Effects of Substrate Affinity on Protein Kinase C Regulation and Inhibitor Potency

Reagents and Constructs—

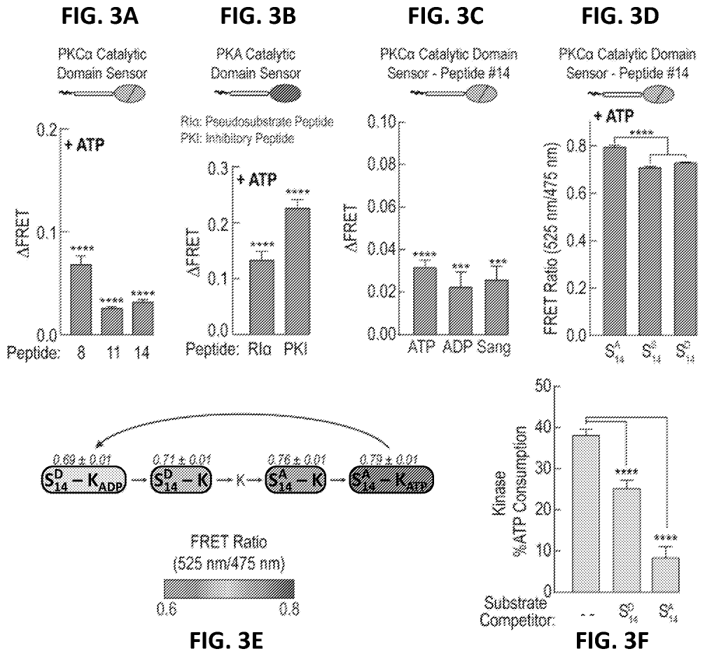

Sangivamycin (Sigma; St. Louis, Mo.) and Bisindolylmaleimide I or BimI (EMD Millipore; Burlington, Mass.) were solubilized in DMSO, ATP (EMD) in 20 mM Imidazole (pH 7.5), and ADP (Sigma) in 50 mM HEPES (pH 7.5). Peptides #1-14 (FIG. 2) were custom-synthesized by GenScript and solubilized in water. All compounds and peptides were aliquotted and stored at either −20° C. or −80° C. to prevent multiple freeze-thaw cycles. Human protein kinase A (PKA) alpha cDNA was purchased from DNASU. PKA sensors contained either the pseudosubstrate peptide from the type Iα regulatory subunit of Protein Kinase A (PKAPep; KGRRRRGAISAEV; SEQ ID NO:1) or a peptide derived from Protein Kinase Inhibitor protein (PKIPep; TTYADFIASGRTGRRNAIHD; SEQ ID NO:2) (FIG. 3B) (Taylor et al., Biochim. Biophys. Acta 1754:25-37, 2005). Human PKCα cDNA was purchased from Open Biosystems as described elsewhere (Swanson et al., J. Biol. Chem. 289:17812-17829, 2014). PKA and PKCα constructs were cloned using PCR (Expand High Fidelity PCR System, Sigma) or site-directed mutagenesis (Pfu-Turbo, Agilent Technologies; Minnetonka, Minn.) into pBiexl (Novagen/Millipore Sigma; Burlington, Mass.) Sf9 expression plasmid vector. For activity and steady-state FRET experiments, sensors contained a monomeric Cerulean (mCer, donor) and monomeric Citrine (mCit, acceptor) FRET pair separated by a 10 nm ER/K single α-helix, as described elsewhere (FIG. 1) (Swanson et al., supra). For time-resolved TCSPC experiments, the ER/K linker instead contained monomeric eGFP (donor) (Altman et al., PLoS Biol. 5:e210, 2007) and monomeric Cherry (mCherry, acceptor).

Insect Cell Expression and Protein Purification—

pBiexl vectors were transiently transfected into Sf9 insect cells cultured in Sf900-II media (Invitrogen/ThermoFisher; Waltham, Mass.) using Escort IV transfection reagent (Sigma) and Optimem I (Life Technologies; Carlsbad, Calif.). Cells were lysed 72 hours post transfection in 20 mM HEPES (pH 7.5), 200 mM NaCl, 4 mM MgCl₂, 0.5% sucrose, 0.5% IGEPAL, 2 mM DTT, 50 μg/ml PMSF, 5 μg/ml aprotinin, and 5 μg/ml leupeptin. Lysate was clarified via high speed centrifugation and incubated with anti-FLAG M2 affinity resin (Sigma) for 1-2 hours. The resin was washed three times with 20 mM HEPES (pH 7.5), 150 mM NaCl, 10 mM MgCl₂, 2 mM DTT, 50 μg/ml PMSF, 5 μg/ml aprotinin, and 5 μg/ml leupeptin. Protein was eluted using FLAG peptide (Sigma), and buffer exchanged into PKC Buffer (20 mM HEPES (pH 7.5), 5 mM MgCl₂, 0.5 mM EGTA, and 2 mM DTT) using 40 kDa cutoff Zeba Spin Desalting Columns (Pierce). Before each experiment, protein samples were centrifuged at ˜220,000×g at 4° C. to remove any insoluble protein. Protein concentration was determined for centrifuged protein from the fluorescent emission of mCit (Ex 490, Em 525 nm) compared to a standard on a FluoroMax-4 fluorimeter (Horiba, Scientific) or by eGFP absorbance on a NanoDrop One (Thermo). For experiments requiring isolated catalytic domain, resin-bound sensors were incubated with TEV protease, before being washed, eluted, and desalted. TEV cleavage was ≥95% as assessed by mCer and mCit fluorescence.

FRET Measurements—

All experiments were performed with 30-50 nM protein in PKC Buffer at 21-22° C. Samples were prepared in tubes pre-coated with 0.1 mg/mL BSA to limit protein sticking to tube walls. Protein samples were excited at 430 nm (mCer) with an 8-nm band pass, and emission monitored from 450 to 650 nm. The FRET ratio was calculated from the ratio of the emission for mCit (525 nm) to mCer (475 nm). Final concentrations of 100 μM ATP, 100 μM ADP, 100 μM Sangivamycin, or BimI were added as indicated. For each experimental condition, ≥3 independent protein batches were used and at least two replicates were measured for each independent experiment (N≥3).

Time-Resolved Measurements—

Time-resolved fluorescence decay measurements were taken by single photon counting (SPC-130-EM, Becker & Hickl) using a 485 nm sub-nanosecond pulse diode laser (PicoQuant), a 519 bandpass filter, and a PMH-100 photomultiplier module (Photonics Solutions) (Autry et al., J. Biol. Chem. 287:39070-39082, 2012). Sensors contained the eGFP and mCherry FRET pair, and all experiments were performed with 50-100 nM protein in PKC Buffer containing 100 μM ATP and 0.1 mg/mL BSA. Data were fit to the second exponential decay, I(t)=A₁e^{-t/τ}^{¹}+A₂e^{-t/τ}^{²}, where τ1 was set to the lifetime of a 10 nm ER/K linker control. Each experimental condition was collected from three independent protein batches (N=3).

Kinase Activity Assay—

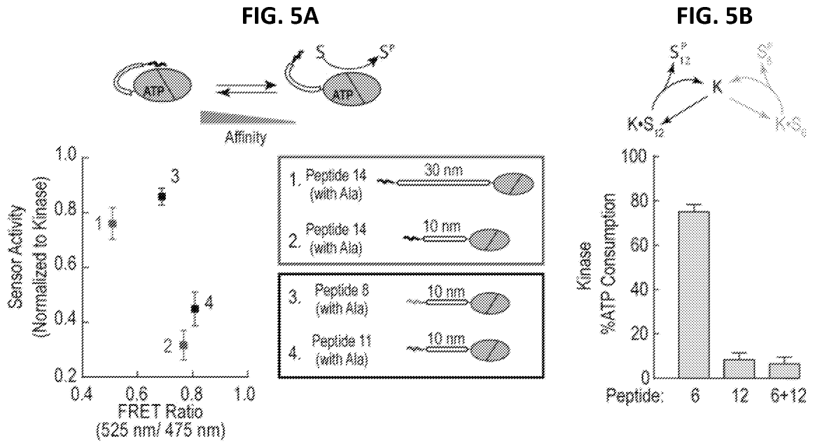

All activity assays were performed with 50 nM isolated catalytic domain in PKC Buffer at 21-22° C. for 2-6 minutes. Experiments comparing the basal activity of different FRET sensors (FIG. 5A) were performed with 50 μM MBP peptide from GenScript. For all other activity experiments (FIGS. 3F, 4B, 5B, and 6B), the final peptide concentration was 500 μM. Activity was measured by monitoring ATP consumption with the Kinase-Glo Max Luminescence Assay Kit (Promega; Madison, Wis.). End-point luminescence was measured in white, 96-well plates using a M5e Spectramax spectrophotometer (Molecular Devices; San Jose, Calif.). For each experimental condition, ≥2 independent experiments were performed for ≥3 protein preparations (N≥6).

### Example 2—Loss of Affinity for Phosphorylated Substrate Enhances Kinase Specific Activity

During catalysis, a kinase must bind both nonphosphorylated substrate and ATP in the active site of its catalytic domain. To be efficient, it is also important that the kinase preferentially selects for non-phosphorylated substrate. To test these transient interactions, three PKCα peptide sensors were examined. Sensors were selected that displayed a range of basal FRET ratios (FIG. 2). Upon addition of 100 ATP μM, there was a significant increase in the peptide interaction with the catalytic domain by steady-state FRET (p<0.001; FIG. 3A). This ATP-dependent substrate interaction appeared to be a wider kinase mechanism, as it also existed for two strong-binding pseudopeptides for PKA (Taylor et al., supra) (FIG. 3B). Next, a sensor containing substrate #14 (which has the sequence NRFARKGALRQKNV (SEQ ID NO:29) and is derived from the pseudosubstrate sequence of PKCα; House and Kemp, Science 238:1726-1728, 1987) was tested, with additional nucleotide-based compounds. In addition to ATP, the peptide-catalytic domain interaction also occurred in the presence of 100 μM of ADP and 100 μM of the non-hydrolysable nucleoside analogue Sangivamycin (Sang; FIG. 3C).

To examine the effects of phosphorylation, the alanine in peptide #14 was mutated to a serine (NRFARKGSLRQKNV; SEQ ID NO:30). Given that this peptide is known to be phosphorylated by PKC (House and Kemp, supra), it was assumed that the serine-containing peptide #14 in the FRET sensor would be phosphorylated in the presence of ATP. Aspartic acid has been successfully used with other PKC substrates to mimic phosphorylation (Schulz et al., Cellular Signalling 25:2485-2495, 2013; Lopez-Sanchez et al., Proc. Natl. Acad. Sci. USA 110:5510-5515, 2013; and Verma et al., FEBS Lett. 582:2270-2276, 2008). Hence, a phosphomimetic version of peptide #14 was also created by mutating the alanine in the peptide to aspartic acid (NRFARKGDLRQKNV; SEQ ID NO:31). In the presence of ATP, both the serine- and the aspartic acid-containing FRET sensors had a significantly lower FRET ratio compared to the alanine-containing peptide (p<0.0001, FIG. 3D). Given the potential heterogeneity in the phosphorylation state of the serine residue, the aspartic acid-substituted peptide was used to mimic the phosphorylation state in studies going forward. With the alanine and aspartic acid-containing substrate #14 sensors, the FRET ratio of the peptide-catalytic domain interaction was measured during different states of the catalytic cycle (FIG. 3E). As compared to alanine-substituted substrate, the catalytic domain had decreased affinity for the phosphomimetic substrate, suggesting a simple mechanism for selecting nonphosphorylated substrate and thereby increasing productive phosphoryl-transfer. To test this preferential substrate selection, ATP turnover of the catalytic domain was monitored for serine-containing peptide #14 in the presence of either equimolar alanine-substituted peptide or the aspartic acid-containing phosphomimetic peptide. Indeed, the alanine-substituted peptide was significantly better at inhibiting catalytic activity (FIG. 3F).

### Example 3—Inverse Correlation Between Substrate Affinity and Kinase Activity

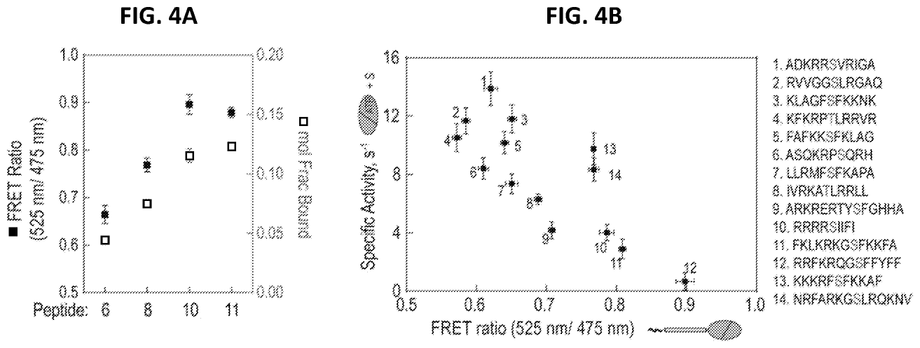

Time-correlated single photon counting (TCSPC) was used to test whether changes in steady-state FRET reflect changes in fractional binding of the peptide to the catalytic domain (FIG. 1). TCSPC is a method for measuring time-resolved fluorescence that provides information on complex decay kinetics and population states (Lakowicz, Principles of Fluorescence Spectroscopy, 3rd ed., Springer, New York, 2006). Using this method, the relative population of interacting to non-interacting species was measured for four peptide sensors displaying a range of steady-state FRET ratios (FIG. 2). As illustrated in FIG. 4A, increases in steady-state FRET strongly correlated with higher fractional binding (see, also, TABLE 1). Interestingly, these data also suggested that the substrate-kinase interaction can occur in the absence of nucleotide.

Next, the affinity of peptide-kinase interaction was compared to the activity of the catalytic domain for that same peptide. As illustrated in FIG. 4B, the steady-state FRET ratio was measured for fourteen peptide sensors in the absence of nucleotide, and plotted versus activity of the isolated catalytic domain in the presence of excess substrate peptide. All activity measurements were taken under the same conditions. A trend was observed in which catalytic activity decreased with increasing basal FRET ratios (i.e., higher peptide binding). This trend was not complete (R²=0.62), as a few peptides displayed similar affinity but significantly different activity (e.g., peptide #1 vs #6, FIG. 4B). This suggests that in addition to affinity, other mechanisms are likely involved in dictating catalytic activity. The data also indicated that the peptide based on the pseudosubstrate of PKCα (#14) bound more strongly than peptides derived from cellular substrates (#1-8; FIG. 4B and FIG. 2). This finding was consistent with the dominant role of the pseudosubstrate in PKC auto-inhibition (House and Kemp, supra).

TABLE 1
Lifetimes and Fractional Binding for Peptide -
Catalytic Sensors

| | Mol Fraction | Mol Fraction | |
|---|---|---|---|
| Peptide | Unbound | Bound | τ2, ns |
| 11 | 0.85 ± 0.01 | 0.15 ± 0.01 | 0.88 ± 0.06 |
| 10 | 0.84 ± 0.01 | 0.16 ± 0.01 | 0.83 ± 0.06 |
| 8 | 0.89 ± 0.01 | 0.11 ± 0.01 | 0.66 ± 0.06 |
| 6 | 0.92 ± 0.01 | 0.08 ± 0.01 | 0.49 ± 0.05 |

Means ± SEM. N = 3 (number of protein preparations)
Data fit to I(t) = A₁e − t/τ₁ + A₂e^{-t/τ}^{²}, where τ₁ is fixed to the lifetime of a donor linker control (2.56 ± 0.01 ns)

### Example 4—High Affinity Substrates Suppresses Phosphorylation of Low Affinity Counterparts

To further examine the trade-off between peptide affinity and activity, the activity of two different sets of peptide sensors for the same substrate was compared. In one set, a sensor was made in which peptide #14 (SEQ ID NO:29) was separated from the catalytic domain by either a 10 nm (SEQ ID NO:32) or a 30 nm (EEEEKKKEEEEKKQKEEQER LAKEEAERKQKEEQERLAKEEAERKQKEEEERKQKEEEERKQKEEEERKLKE EQERKAAEEKKAKEEAERKAKEEQERKAEEERKKKEEEERLERERKEREEQE KKAKEEAERIAKLEAEKKAEEERKAKEEEERKAKEEEERKKKEEQERLAKEK EEAERKAAEEKKAKEEQERKEKEEAERKQR; SEQ ID NO:33) ER/K linker. By increasing the linker length, the effective concentration of the peptide for the catalytic domain was changed from ˜10 μM to 100 nM (Sivaramakrishnan and Spudich, supra). Reducing the effective concentration should weaken the apparent peptide affinity, as observed by a decrease in the FRET ratio, and result in a higher basal activity of this sensor. Indeed, the sensor with the 30 nm linker had significantly higher activity than the 10 nm linker for the same substrate peptide (FIG. 5A). For a second set of sensors, the activity of two different 10 nm peptide FRET sensors was compared for the same substrate. Sensors contained either a high affinity alanine-peptide (#11; SEQ ID NO:23) or a lower affinity alanine peptide (#8; SEQ ID NO:17). In agreement with the previous set, the higher affinity peptide sensor (#11) had a much lower activity than the lower affinity peptide sensor (#8) (FIG. 5A). Finally, the tradeoff between affinity and activity was tested by examining the effect of a high affinity-low activity peptide (#12; SEQ ID NO:26) on catalytic turnover with a low affinity-high activity peptide (#6; SEQ ID NO:14). Concentrations of both substrates were held constant, and indeed, when both peptides were present, the activity was essentially inhibited to levels matching the high affinity-low activity peptide (FIG. 5B). This suggested a new model of substrate selectivity in which high-affinity interactions may out-compete low affinity, high activity substrates.

### Example 5—Kinase-Substrate Binding Affinity Impacts Inhibitor Potency

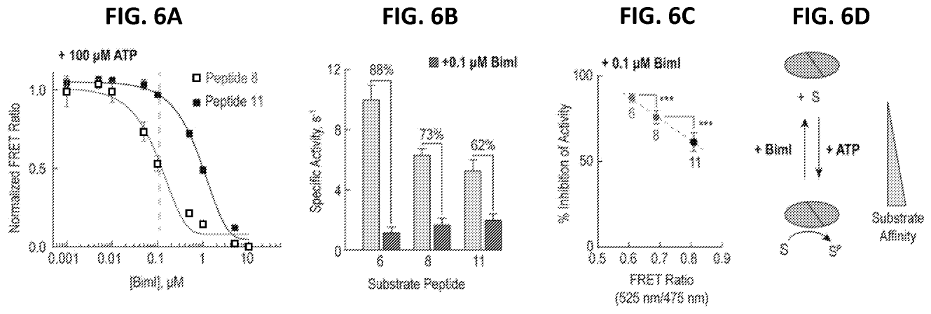

As PKC appears to play a role in numerous diseases, one of the goals of therapeutic drug discovery is to develop selective modulators of PKC activity (Mochly-Rosen et al., Nature Rev Drug Discov. 11:937-957, 2012). These include ATP competitive inhibitors such as Sangivamyicin (FIG. 3C) and substrate-competitive inhibitors such as Bisindolylmaleimide I or BimI (Smith and Hoshi, PloS One 6:e26338, 2011). Using a high-affinity (#11; SEQ ID NO:23) peptide sensor and a low affinity (#8; SEQ ID NO:17) peptide sensor, the effect of BimI on the ATP-dependent peptide-kinase interaction was examined. These peptides were selected since they display significantly different basal FRET ratios but also provide a dynamic FRET range for the BimI titration. In the presence of BimI, the substrate-kinase interaction was indeed abrogated as measured by steady-state FRET (FIG. 6A). Higher concentrations of BimI, however, were required to disrupt the higher-affinity peptide interaction (black, FIG. 6A). This was further reflected when comparing the percent inhibition of catalytic activity for three different substrate peptides (order of affinity: 11 (SEQ ID NO:24)>8 (SEQ ID NO:18)>6 (SEQ ID NO:14); FIGS. 6B and 6C). Overall, this demonstrated that the potency of a substrate-competitive inhibitor is strongly influenced by the strength of the substrate-kinase interaction (FIG. 6D).

### Example 6—Effect of Various Kinase Inhibitors on Substrate Binding

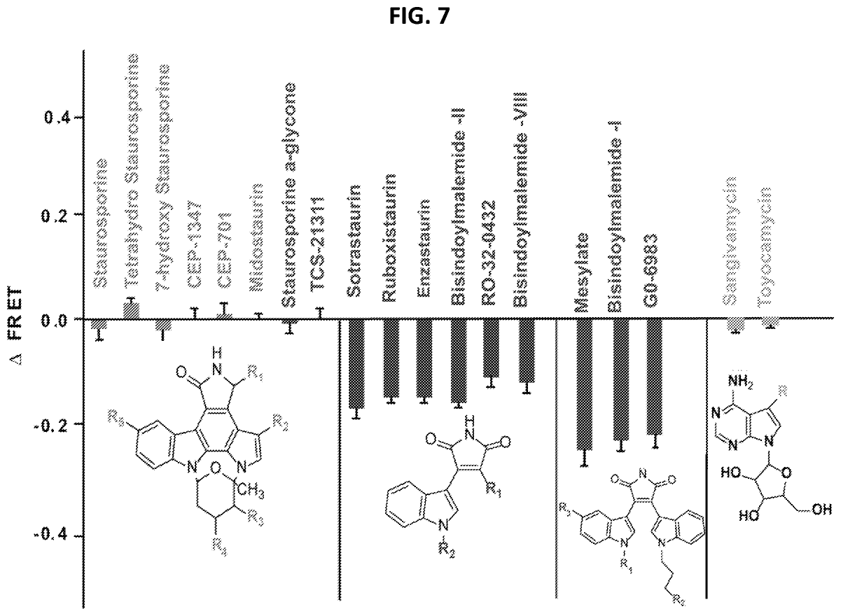

A drug screen was carried out using different types of kinase inhibitors on the interaction between the PKCα catalytic domain and the EGFR substrate peptide (SEQ ID NO:17) as quantified by the change in FRET ratio (ΔFRET) between untreated and inhibitor treated conditions. As shown in FIG. 7, compounds that are structurally similar to Bisindolylmaleimde-I (BimI) had the greatest influence on FRET ratio compared to nucleotide analogs (Sangivamycin and Toyocamycin) and staurosporine-like compounds. These data demonstrate the ability of sensors to differentiate between kinase inhibitors based on their effects on substrate binding. Inhibitors that can displace both substrate and ATP binding to the kinase can serve as bi-topic ligands that can modulate the cellular activity of the kinase with higher potency and specificity relative to other kinases or substrates.

### Example 7—Experimental Procedures: Assessing the Structural Mechanisms Determining Substrate Affinity and Kinase Activity of Protein Kinase Cu

Computational Methods

Homology Modeling of the PKCα Kinase Structure—

The template structure closest to PKCα is that of PKC βII with the ANP bound (PDB ID 3PFQ)(Leonard et al., Cell 144:55-66, 2011) and hence this was used for the homology modeling of PKCα with Modeller 9 (Webb and Sali, Curr. Protoc. Bioinforma. 2014:5.6.1, 2014). Out often models retained from Modeller, the top scoring (by DOPE score in Modeller) homology model of the PKCα was selected, and the binding of the 14 peptide substrates shown in TABLE 2 was modeled. The starting coordinates for each peptide substrate were obtained from the crystal structure of PKB (PDB ID 106L) (Yang et al., Nat. Struct. Biol. 9:940-944, 2002) that has a peptide with 10 amino acids bound. The PKCα homology model was aligned to the crystal structure of PKB and then the peptide was transferred from PKB by retaining the contact between the phosphorylated Ser in the peptide to Asp466 in PKCα (which is D275 in PKB crystal structure). The residues in the peptide transferred from PKB were mutated to their appropriate amino acid sequences shown in TABLE 2 using the mutation function in Maestro (Schrodinger Inc., San Diego, Calif.). Most of the peptides in TABLE 2 are longer than that in the PKB crystal structure. Longer peptides were grown by adding residues to the N- or C-terminus using loopModel.pl script in Modeller. After the entire peptide was grown, the side chain conformations of residues were optimized within 5 Å of each amino acid residue in the peptide, taking into account the residues that are common to the neighborhood of one or more residues in the peptide. The Prime module in the Maestro program (Jacobson Et al., Proteins Struct. Funct. Genet. 55:351-367, 2004) was used for side chain repacking, and the whole complex was minimized in energy with the PRCG (Polak-Ribier Conjugate Gradient) method using Macromodel. The forcefield parameters for the initial fourteen PKC-substrate complexes with ATP bound for MD simulations were assigned using the CHARMM package (Brooks et al., Comput. Chem. 30:1545-1614, 2009). All starting complexes were solvated using TIP3P water molecules (Beglov and Roux, J. Chem. Phys. 100:9050-9063, 1994) and neutralized to an ionic strength of 0.15 M by adding Na⁺ and Cl⁻ ions. The system was then minimized in energy, heated from 0 to 310 K using the default settings in the simulated annealing module of GROMACS, and equilibrated by performing 5.2 ns of MD at 310 K using NVT ensemble followed by 55 ns of MD under NPT ensemble with pressure at 1.0 atm. During the first 35 ns of the equilibration, position restraints were placed to the atoms in PKCα, the peptide, and ATP, and the restraints were gradually decreased from 5 kcals/mol to 0 kcal/mol in steps of 1 kcal/mol. A hydrogen bond distance restraint also was placed from ATP to K350, E421, and V423 in its binding site. In addition, a distance restraint was used from the Ser/Thr of the peptide to D466 in the C-lobe of PKCα. All restraints were removed for the last 20 ns of the equilibration simulations. All equilibration simulations for the 14 peptides bound to PKCα were performed using GROMACS5.1.0 and the CHARMM36 forcefield (Huang and MacKerell, J. Comput. Chem. 34:2135-2145, 2013). A net charge of −2.0 was used for ATP. The final complex structures obtained after equilibration were used for further simulations using GNEIMO torsional MD simulation.

Optimization of Peptide Binding Conformation Using GNEIMO-REMD Dynamics Simulations—

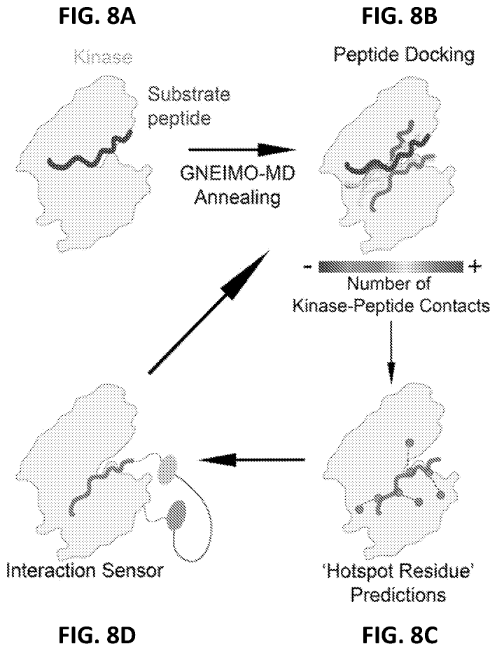

The GNEIMO method is an internal coordinate MD method, and when the bond lengths and bond angles are treated as rigid holonomic constraints, GNEIMO performs torsional MD simulations (Jain et al., J. Comput. Phys. 106:258-268, 1993; Vaidehi et al., J Phys. Chem. 100:10508-10517, 1996; Balaraman et al., J Phys. Chem. B. 115:7588-7596, 2011; and Park et al., J Phys. Chem. B. 116:2365-2375, 2012). A schematic of the workflow combining the GNEIMO with SPASM FRET methods to probe the dynamics of the kinase peptide substrate interactions is shown in FIGS. 8A-8D.

The GNEIMO torsional MD method also was combined with the replica exchange method (REMD) (Sugita and Okamoto, Chem. Phys. Lett. 314:141-151, 1999) to enhance the conformational sampling, as the GNEIMO-REMD combination is effective for folding small protein structures (Wagner et al., J. Comput. Chem. 34:904-914, 2013) and for refining homology models of proteins (Park et al., supra; Larsen et al., Chem. Inf. Model. 54:508-517, 2014; and Larsen et al., J. Comput. Chem. 35:2245-2255, 2014). GNEIMO software (GneimoSim, Larsen et al., 2014b, supra) was combined with Rosetta software for combining the advantages of torsional Monte Carlo with torsional MD simulations, and to use the side chain packing algorithms in Rosetta along with torsional MD simulations of GNEIMO for protein structure refinement. The GNEIMO-REMD-ROSETTA protocol was developed for performing annealing torsional MD simulations. This protocol, as described below, was efficient for protein structural refinement and for generating an ensemble of the structures for peptide-bound PKCα.

In these studies, a multi-scale MD protocol was developed to optimize the binding site to PKCα of the 14 peptides shown in TABLE 2. GNEIMO-REMD was used with Rosetta (online at rosettacommons.org) as a coarse grain dynamic model to anneal and optimize the binding of the 14 peptides in PKCα. An advantage of GNEIMO is that the backbone of secondary structure elements, such as helices or beta sheets, can be treated as rigid bodies while sampling the side chain and other loop regions as flexible torsions during the dynamics. The backbone and side chain torsion angles of residues within 5 Å of ATP and the peptide were all treated as flexible torsions during the GNEIMO torsional MD simulations.

Integration of GNEIMO with Rosetta—

The use of GNEIMO simulations and physical forcefields can enhance conformational sampling and protein structure refinement (Larsen et al. 2014a, supra; Gangupomu et al., Biophys. J. 104:1999-2008, 2013; and Park et al., supra). The Rosetta software suite has many functionalities that perform well consistently for protein structure refinement and side chain repacking (Park et al., Proteins Suppl. 1:314-322, 2016). GneimoSim software (Larsen et al. 2014b, supra) has been combined with the Rosetta software to (1) perform torsion MD simulations with Rosetta forcefield (Alford et al., J. Chem. Theor. Comput. 13:3031-3048, 2017) and (2) enhance conformational sampling during protein structure refinement by combining the benefits torsional MD in GNEIMO with the torsional Monte Carlo sampling and side chain repacking in Rosetta using a rotamer library. Details of the software integration can be found elsewhere (Larsen et al. 2014b, supra).

To perform GNEIMO-REMD-Rosetta annealing simulations, the side chain conformations were first repacked in the peptide-PKCα complex built by homology modeling, using the PackRotamersMover module in Rosetta. The resulting structures were minimized using the lbfgs_armijo_nonmonotone minimizer in the CartesianMinimizer module in Rosetta. The starting structures of the 14 different peptides bound PKCα had the peptide in the extended conformation, except for the p-Ser/Thr making contact with the Asp466 in the kinase. To optimize the structure of the peptide binding to PKCα, 2000 to 2500 annealing cycles were performed for each peptide-PKCα pair using GNEIMO-REMD-Rosetta torsion MD simulations as described below. The Rosetta module was used for the forcefield and side chain repacking after each annealing cycle. The side chain repacking combined with GNEIMO torsional MD enhanced the conformational sampling of the peptide substrate binding to PKCα. Each annealing cycle consisted of a side chain rotamer repacking of all the residues in the peptide-kinase complex using the Rosetta PackRotamersMover module and an all-atom minimization using the CartesianMinimizer, followed by the GNEIMO-REMD-Rosetta torsion MD simulation run with 12 replicas. The range of temperature used in REMD was 200 to 300 K for a total of 12 replicas with an integration step size of 1 fs. The Talaris 2014 with the Lazaridis-Karplus implicit solvent model was used with a distance dependent dielectric function, a variant of the Lobatto integrator within the GNEIMO module (Park et al., supra), and 0.5 ps of Nose-Hoover constant (t) at constant temperature.

The conformation ensemble generated from all the annealing cycles for each peptide-PKCα pair was clustered using RMSD-based clustering with a cut-off of 1.5 Å. The number of hydrogen bonds and van der Waals contacts the peptide makes with PKCα was then calculated for every conformation in the most populated cluster in the ensemble. The conformation with the maximum number of hydrogen bonds and van der Waals contacts to PKCα was extracted from the most populated cluster and used as the starting structure for performing all-atom MD simulations in explicit solvent with GROMACS module.

All Atomic MD Simulations—

All-atom MD simulations were performed in explicit solvent for each of the 14 peptides bound to PKCα. The starting conformations of the PKC complex with ATP and the peptide substrates for the all-atom MD simulations were obtained as follows. The conformations from the GNEIMO-REMD simulations were clustered by root mean square deviation (RMSD) of the main atoms. From the most populated cluster of conformations, the conformation that showed the maximum number of favorable peptide-kinase interactions was chosen for each of the 14 peptides. To this starting conformation, hydrogens were added, and the structures were solvated in the explicit TIP3P water molecules (Beglov and Roux, supra). MD simulations on PKC with periodic boundary conditions were performed using the GROMACS package (Van Der Spoel et al., J. Comput. Chem. 26:1701-1718, 2005) with CHARMM36 forcefield (Huang and MacKerell, supra). The LINCS algorithms (Hess et al., J. Comput. Chem. 18:1463-1472, 1998) were used for the bond and angle for water and all other bonds, allowing 2 fs of time step. For the analysis, the coordinates were saved every 2 ps. A cutoff distance of 12 Å for nonbond interactions was introduced, and the PME (particle mesh Ewald) method (Darden et al., J. Chem. Phys. 98:10089-10092, 1993; and Essmann et al., J. Chem. Phys. 103:8577-8593, 1995) was used for long-range Van der Waals interactions. MD simulations were performed on fourteen systems, each 100 ns long. Each of the fourteen systems were heated slowly and equilibrated by performing 5 ns of MD at 310 K using a NVT ensemble followed by the MD under NPT conditions with a pressure of 1 bar, with initial velocities sampled from the Boltzmann distribution and with 5 kcal/mol/Å2 harmonic position restraints applied to all non-hydrogen atoms of the protein and peptide substrate and distance restraints applied to crucial residues of peptide substrate and kinase receptor. The position and distance restraints were linearly tapered over the 55 ns of equilibration period. After equilibration to the expected temperature and pressure, a total of three production simulations of up to 200 ns were performed for each initial conformation with different initial velocities using the NPT ensemble. For the trajectory analysis, last 100 ns of trajectory per each simulation from the MD simulations were considered using tools provided by GROMACS and Python script.

Analysis of Catalytic Conformations—

To quantify the activity of the PKCα toward several substrates, the distances between the phosphorous atom of the γ-PO₄ group of ATP and the side chain oxygen atom of Ser/Thr of the substrate peptide were analyzed. In addition, the distance between the side chain oxygen atom of Ser/Thr of the substrate peptide and the oxygen atom of Asp466 of the PKCα was calculated. The Asp466 is known to be involved in catalysis and proton abstraction from the hydroxyl group of the Ser/Thr of the substrate peptide. The conformations from all-atom MD simulations for each peptide were clustered by these two distances to identify which peptide substrates lead to an enrichment of the catalytic conformations. These distances are 4.4 Å and 2.5 Å, respectively, in the crystal structure of ATP bound with the CP20 peptide in PKA (PDB ID 4XW5) (Gerlits et al., J. Biol. Chem. 290:15538-15548, 2015), in which the Ser of the SP20 peptide substrate is mutated to Cys. This crystal structure captures the optimal distances required for direct phosphoryl catalysis to occur, and call this conformation is referred to as the “catalytic conformation.”

MD Simulations of PKCα after Phosphoryl Transfer—

The catalytic conformation nearest to the crystal catalytic conformation from the all-atom MD simulations was extracted for each of the 14 substrates bound PKCα, and using this conformation, the PO₄ group was transferred from ATP to the Ser/Thr of each peptide. To understand the PKCα dynamics after phosphoryl transfer from ATP to the Ser/Thr of the 14 substrate peptides, the phosphate group was transferred to the Ser/Thr of the substrate peptide, and MD simulations of PKCα were initiated. All-atom MD simulations were conducted for PKCα bound to ADP and p-Ser/Thr peptide substrates. In the peptide substrate, the amino acid Ser9 was converted to phosphorylated Ser9. The patch SP2 in the CHARMM 36 forcefield was used to assign forcefield parameters for p-Ser and p-Thr. Using the same MD simulation conditions, all atom MD simulations were performed, solvated with explicit TIP3 water with periodic boundary conditions using the GROMACS package with the CHARMM36 forcefield. MD simulations were performed for high activity peptide p1 for 100 ns.

Analysis of GNEIMO and all Atomic MD Trajectories

RMS Deviation and Fluctuation—

To compare the inherent flexibility and dynamics of proteins as generated by GNEIMO simulations, the RMSF relative to the average structure for each residue were calculated. RMSDs and RMSFs were calculated using the gmx rmsf utilities in the GROMACS MD package (Van Der Spoel et al., supra).

Binding Affinity Calculation—

To estimate the binding energy of small peptide substrate to kinase receptor, the interaction energy between the N-terminus of each peptide and the kinase was calculated using the gmx energy in the GROMACS MD package (Van Der Spoel et al., supra). The interaction energy of PKCα for the peptide substrates was determined by the non-bonded energies, short range Coulomb, and Lennard-Jones energies.

RMSD-Clustering—

To obtain the representative conformation from the most populated conformational clusters, RMSD-based clustering was used to cluster all the conformations within the most populated cluster from the molecular dynamics trajectories. A RMSD cut-off of 1.5 Å was used and a representative snapshot was taken from the closest rms deviation from the average structure of the most populated clusters.

Inter-Molecular Contact—

For inter-molecular hydrogen bond analysis using 3.5 Å and 30° for the cut-off distance and angle, respectively, the gmx_hbond utility of GROMACS was used. Inter-molecular hydrogen bond interactions were derived from the stable hydrogen bond criteria having more than 50% occupancy (population) through all trajectories, which were normalized by setting the most densely populated point to 1. For the inter-molecular hydrophobic (Van der Waals) interactions over the whole trajectories with more than 50% of duration during MD simulations, contactFreq of the Tcl script was used.

Other

Reagents and Peptides—

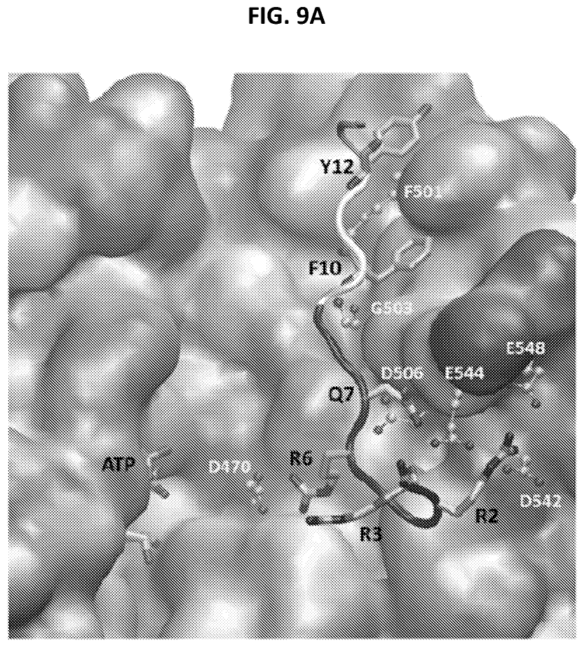

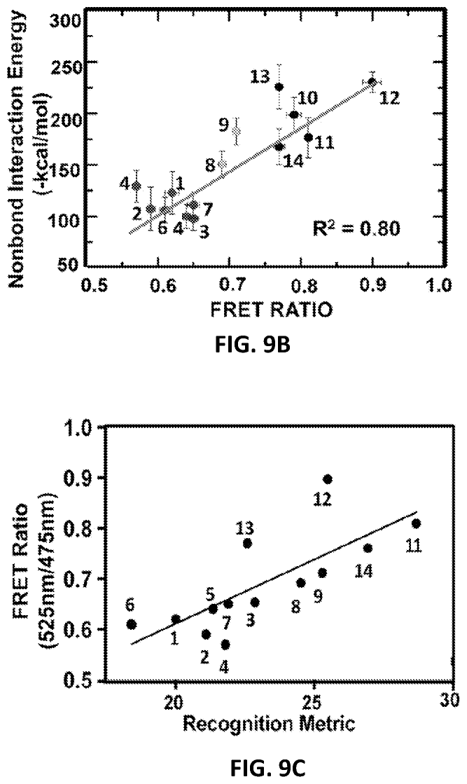

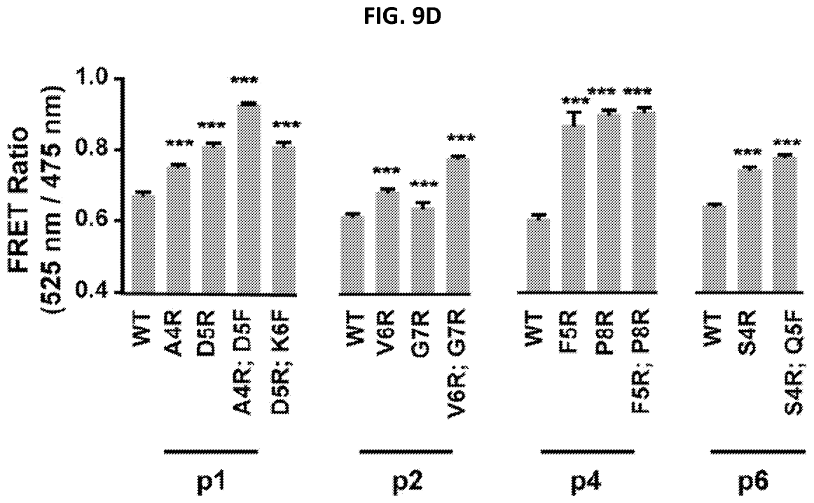

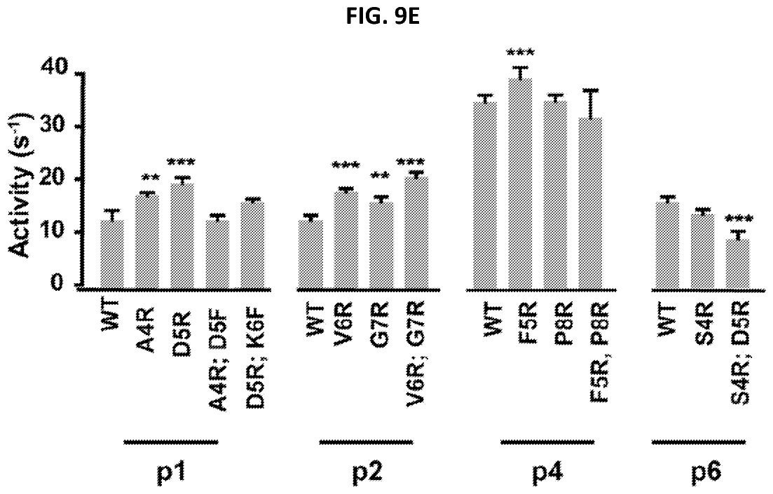

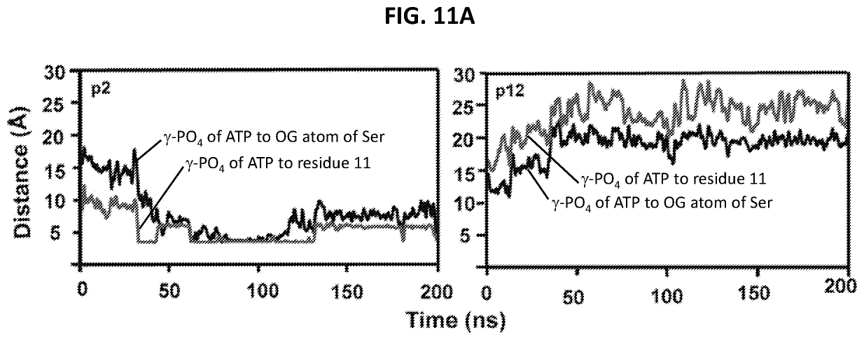

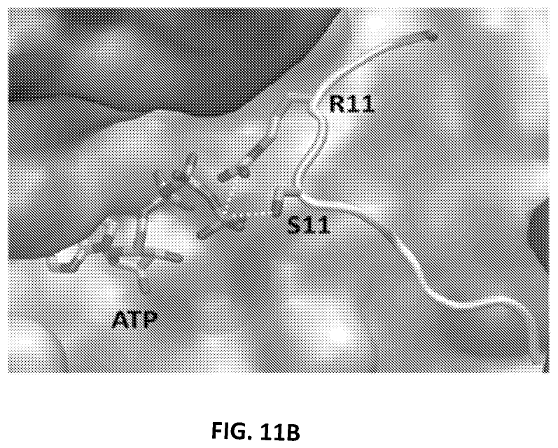

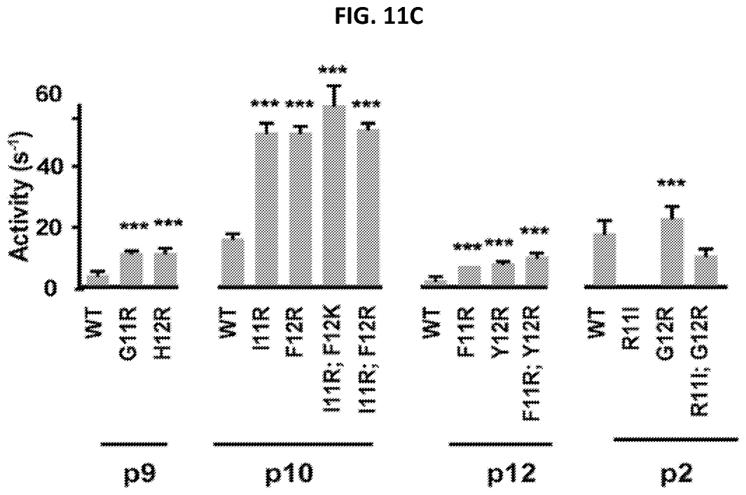

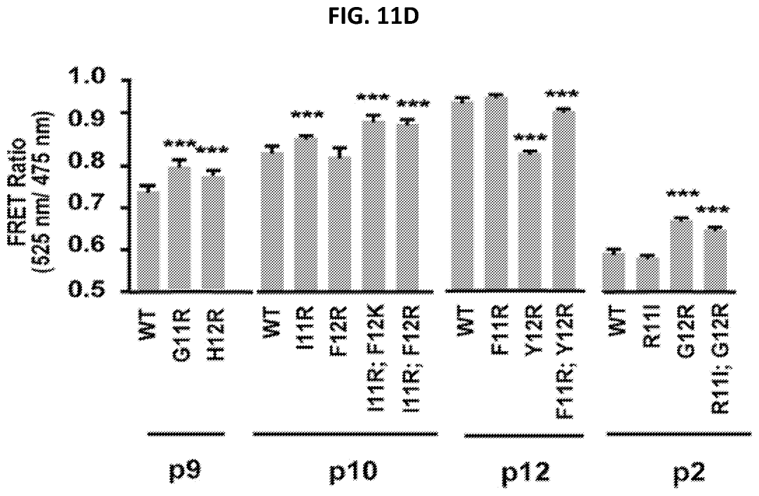

Buffer chemicals were purchased from various commercial vendors and used without further purification. Peptides (1, 2, 4, 6, 9, 10 and 12) and the corresponding mutant peptides (FIGS. 9D, 9E, 11C, and 11D) were custom-synthesized by GenScript and solubilized in PKC Buffer (20 mM HEPES pH 7.5, 5 mM MgCl₂, 0.5 mM EGTA, and 2 mM DTT).

Constructs—

The human PKCα catalytic domain, mCerulean (FRET donor), mCitrine (FRET acceptor), 10 nm ER/K single α-helix and Ala-substituted substrate peptides, were PCR amplified and cloned into unique restriction sites in the pBiexl expression vector (Novagen). Gly-Ser-Gly linkers were inserted between protein domains to allow for rotational freedom. Mutant peptides were introduced into sensor by digestion, followed by phosphorylation and ligation of appropriate phosphorylated oligonucleotides. Sanger sequencing was used to confirm appropriate insertion and mutagenesis.

Insect Cell Expression and Protein Purification—

Plasmids were transfected into (Sf9) insect cells using a protocol reported elsewhere (Sommese and Sivaramakrishnan, J. Biol. Chem. 291:21963-21970, 2016). Briefly, pBiexl vectors were transiently transfected into Sf9 insect cells cultured in Sf900-II media (ThermoFisher) using Escort IV transfection reagent (Sigma) and Optimem 1 (ThermoFisher). Cells were harvested by centrifugation (250 g, 5 minutes at 4° C.) and lysed using lysis buffer (20 mM HEPES pH 7.5, 200 mM NaCl, 4 mM MgCl₂, 0.5% sucrose, 0.5% IGEPAL, 2 mM DTT, 50 μg/ml PMSF, 5 μg/ml aprotinin, and 5 μg/ml leupeptin). Clarified lysate after ultracentrifugation (13435 g, 25 minutes at 4° C.) was incubated with anti-FLAG M2 affinity resin (Sigma) for 1-2 hours and washed with wash buffer (20 mM HEPES pH 7.5, 150 mM NaCl, 10 mM MgCl₂, 2 mM DTT, 5 μg/ml PMSF, 5 μg/ml aprotinin, and 5 μg/ml leupeptin). Proteins were eluted with FLAG peptide (Sigma, 100 μg/ml) and buffer exchanged to PKC buffer (20 mM HEPES pH 7.5, 5 mM MgCl₂, 0.5 mM EGTA, and 2 mM DTT) using Zeba Spin Desalting Columns (40 kDa, Pierce). The concentration of centrifuged protein was determined from the fluorescent emission of mCit (Ex 490, Em 525 nm) compared to a standard on a FluoroMax-4 fluorimeter (Horiba Scientific; Edison, N.J.) or by fluorophore absorbance on a NanoDrop One (ThermoFisher).

Kinase Activity Assay—

Kinase specific activity was inferred from the ATP consumed in phosphorylation reactions using the KINASE-GLO® Max Luminescence Assay Kit (Promega; Madison, Wis.). ATP depletion was monitored with the KINASE-GLO® Reagent, which uses luciferin, oxygen, and ATP as substrates in a reaction that produces oxyluciferin and light. The luminescent signal is linearly proportional to the amount of ATP remaining following the phosphorylation reaction. Control experiments in which the substrates were incubated with the assay buffer and then assayed with the luciferase showed that substrate/buffer interactions with the luciferase reaction were insignificant under the assay conditions.

Activity assays were performed using the catalytic domain (25-75 nM) and 500-μM peptide in PKC Buffer. Reactions were initiated by the addition of 250 μM ATP to a total reaction volume of 80 μL in U-bottom, white 96-well plates. Following a 3 minute incubation at 22° C., reactions were quenched and subsequent steps were performed as per instructions from the kit provider. End-point luminescence was measured in a FLEXSTATION® 3 plate reader (Molecular Devices). Control experiments with only the kinase or the substrate showed negligible ATP consumption. For each experimental condition, ≥2 independent measurements were performed for ≥3 protein preparations (N≥6).

Steady-State FRET Measurements—

Protein-protein interactions in live cells using SPASM FRET sensors have been extensively monitored as described elsewhere (Sivaramakrishnan and Spudich, supra; Swanson et al., supra; and Malik et al., J Biol. Chem. 288:17167-17178, 2013). FRET measurements provide a direct measure of the binding strength of the substrate peptides to the kinase catalytic domain (Sivaramakrishnan and Spudich, supra; and Sommese et al., J. Biol. Chem. 292:2873-2880, 2017). Before each experiment, purified sensors were centrifuged (17645 g, 4° C.) to remove any insoluble protein. All experiments were performed with 50 nM protein in PKC Buffer at 21-22° C. Samples were prepared in tubes pre-coated with BSA (0.1 mg/ml) to limit protein loss through adsorption to tube walls. FRET measurements were performed using FluoroMax-4 fluorometer (Horiba Scientific). Sensors were loaded in a quartz cuvette (1 cm path length) and excited at 430 nm with an 8-nm band pass, and emission was monitored (4-nm band pass) from 450 to 650 nm. The FRET ratio was calculated from the ratio of the emission for mCitrine (525 nm) to mCerulean (475 nm). For each experimental condition, ≥2 experimental replicates were measured for ≥3 independent protein batches (N≥6).

TABLE 2
Amino acid sequences of peptide substrates (position 9 contains phosphorylated Ser or Thr)

| | | | | | | | | | | | | | | | | SEQ ID | |
|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | NO: | Protein |
| P1 | | | | A | D | K | R | R | S | V | R | I | G | A | | 4 | Kit/SCRF |
| p2 | | | | R | V | V | G | G | S | L | R | G | A | Q | | 6 | PTP1B |
| P3 | | | | L | A | G | G | F | S | F | K | K | N | K | | 8 | MARCKS |
| p4 | | | | K | F | K | R | P | T | L | R | R | V | R | | 10 | Troponin |
| P5 | | | | F | A | F | K | K | S | F | K | L | A | G | | 12 | MARCKS |
| p6 | | | A | S | Q | K | R | P | S | Q | R | H | | | | 14 | Myelin Basic Protein |
| P7 | | | | L | L | R | M | F | T | K | A | P | A | | | 34 | GABA Type A Receptor |
| p8 | | | | I | V | R | K | A | T | L | R | R | L | L | | 18 | EGF Receptor |
| p9 | A | R | K | R | E | R | T | Y | S | F | G | H | H | A | | 20 | Optimized AKT Sub. |
| p10 | | | | | R | R | R | R | S | I | I | F | I | | | 22 | Optimized PKA Sub. |
| p11 | | F | K | L | K | R | K | G | T | F | K | K | F | A | | 35 | Optimized PKCβ Sub. |
| p12 | | R | R | F | K | R | Q | G | S | F | F | Y | F | F | | 26 | Optimized PKCζ Sub. |
| p13 | | | | K | K | K | R | F | S | F | K | K | A | F | | 28 | MARCKS |
| p14 | | N | R | F | A | R | K | G | T | L | R | Q | K | N | V | 36 | PKCα Pseudosubstrate |

### Example 8—the Residues in the N-Terminus of the Peptide Substrates Contribute to Substrate Binding in PKCα

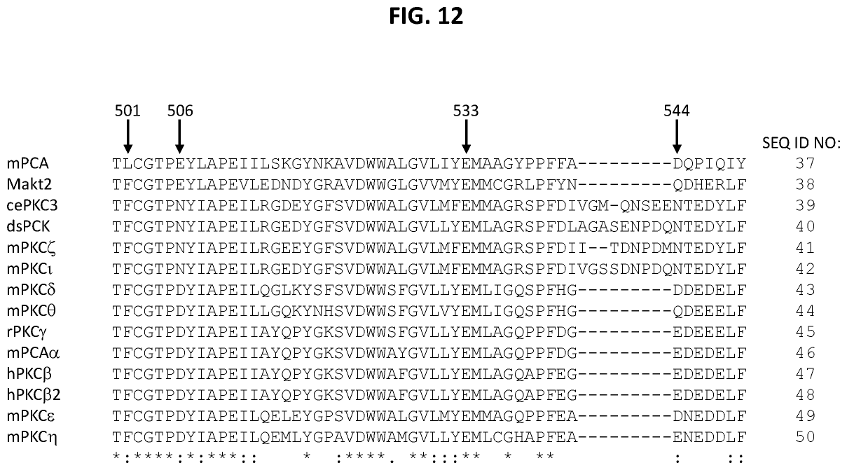

The dynamics of 14 peptide substrates binding to the catalytic domain of PKCα were studied using GNEIMO coarse grain MD simulations, followed by all-atom MD simulations. FIG. 9A shows the binding groove of the tight-binding peptide, p12, that shows lowest kinase activity among the 14 peptides (see FIG. 4B for the relative binding affinities and kinase activity of all 14 substrate peptides). The MD simulation results indicated that an acidic patch of residues located in the substrate-binding groove of PKCα make strong electrostatic interactions with the basic residues in the N-terminus of the peptide substrates (FIG. 9A). In contrast, the residues in the kinase that interact with the C-terminus of the peptide are hydrophobic, resulting in a deep insertion of the peptide C-terminus (FIG. 9A). The residues in PKCα that make sustained (present in over 40% of the snapshots from MD simulations) salt bridge, hydrogen bond, or van der Waals contact with residues in the N-terminus of the peptides were analyzed. The acidic patch of residues consisting of D383, D470, D506, E533, and E544, E548 and D542 (shown in spheres and sticks in FIG. 9A) make close contact with the N-terminus residues of the peptide substrates. Many of these acidic residues listed above are conserved across the PKC family, as shown in FIG. 12.

The stronger electrostatic interactions of the peptide N-terminus compared to the C-terminus suggested that this region contributes substantially to the peptide binding energy. To test this possibility, the average interaction energies, averaged across the three all-atom MD trajectories for each peptide substrate, were calculated and compared to previous FRET measurements (Sommese and Sivaramakrishnan, supra). The calculated interaction energies between the N-terminus residues of the peptide substrate with the catalytic domain of PKCα correlate linearly with the measured binding affinity of kinase-substrate peptide interaction (Sommese and Sivaramakrishnan, supra; and Sivaramakrishnan and Spudich, supra) with a R²=0.8, as shown in FIG. 9B. However, the calculated interaction energies of the entire peptide substrate with the catalytic domain of PKCα did not show a good correlation (R²=0.48).

The structural ensembles for the peptide-PKC interface interactions were generated using GNEIMO-MD simulations followed by all-atom MD simulations as described in Example 7. To test these predicted peptide-kinase interactions, mutations were proposed in poor binding and high activity peptides such as p1, p2, p4, and p6 to improve their binding affinity to the kinase without affecting the kinase activity toward these peptides. The average interaction energy of each residue in every peptide substrate with the kinase was calculated. Using the residues that contribute weakly to the substrate-kinase interaction, mutations were predicted in these four weak binding peptides, and mutants were expressed as FRET sensors along with the catalytic domain of PKCα. Residue numbering on the peptide substrates was from the N-to-C terminus, with the phosphorylated Ser/Thr numbered as position 9 for all peptide substrates (see, TABLE 2). Each mutation showed a significant increase in FRET ratio, suggesting an increase in binding affinity of the peptide for the catalytic domain (FIG. 9D). It was clearly observed that mutation of N-terminal residues to a basic residue such as Arg increased affinity for the acidic patch of PKCα, and therefore improve binding.

As seen in FIG. 9E, most of the mutations in the N-terminus residues of p1, p2, p4, and p6 showed a small but not significant increase in kinase activity. This suggested that the kinase activity on these 14 peptide substrates may be governed additionally by other mechanisms. Interestingly, the kinase-substrate binding affinity correlated linearly with a recognition metric derived based on the consensus motif (FIG. 9C). As described elsewhere (Nishikawa et al., supra), the substrate recognition motif of PKCα was characterized using a degenerate peptide library phosphorylated by the kinase. For each location relative to the phosphorylated serine/threonine, the fold enrichment in substrate phosphorylation with a specific amino acid was compared to the same position of a degenerate sequence. With the assumption that the reported fold enrichments are additive, these values were linearly combined for all conserved residues to arrive at a recognition metric. The finding described herein highlights the role of the consensus motif and its correlation to the peptide substrate binding affinity, and not to kinase activity. The properties of the dynamics of the peptide substrate that would correlate to the kinase activity were examined further, revealing that peptide substrates with good kinase activity showed an enrichment of catalytic conformations during the dynamics simulations.

Recently, crystal structures of PKA representing different snapshots of the kinase in the catalytic process of transferring the PO₄ group were solved, showing a plausible “catalytic conformation” of PKA (Gerlits et al., Biochem. 52:3721-3727, 2013). Such a catalytic conformation shows the Ser/Thr that is phosphorylated in the peptide substrate close to Asp166 (residue number as in PKA), as well as the γ-PO₄ group of the ATP. The Asp166 is involved in abstracting the proton from the hydroxyl group of the p-Ser, enabling transfer of the PO₄ group from ATP to p-Ser/Thr in the peptide substrate. This definition of the catalytic conformation was used to analyze the differences in the population of the “catalytic conformation” in the conformational ensemble derived from MD trajectories for the high activity peptides p1, p2, and p3, compared to the poor activity peptides p10, p11, and p12.

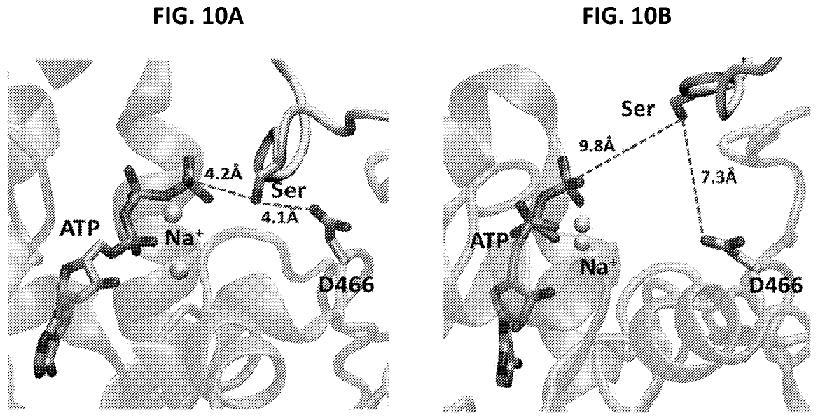

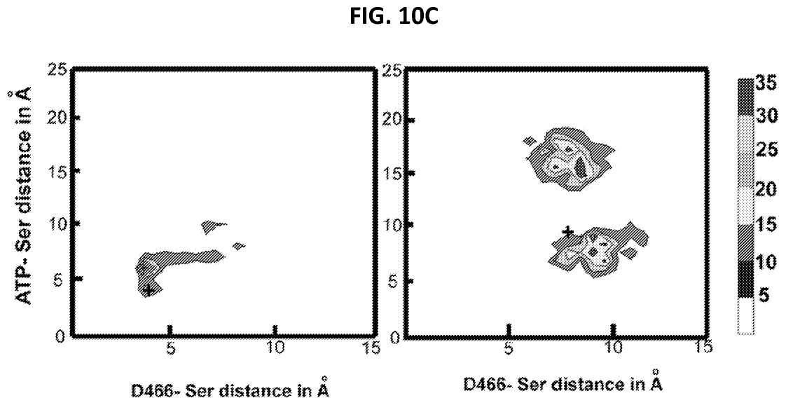

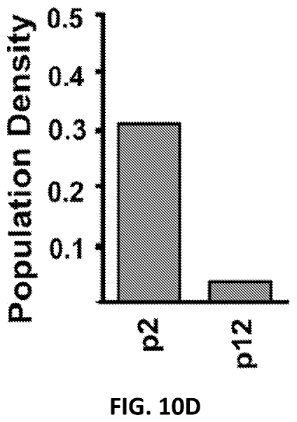

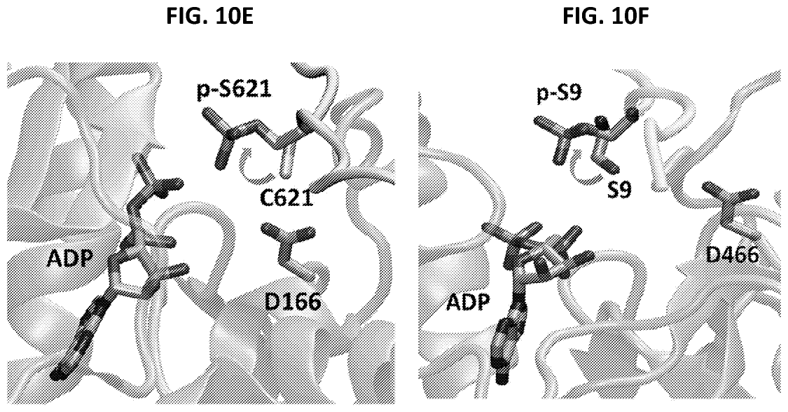

FIG. 10A is a snapshot from the MD simulation trajectory of high activity peptide p2, showing the shortest distance between the Ser in p2, D466 of PKCα, and the γ-PO₄ group of ATP. This conformation is representative of a plausible catalytic conformation of PKCα as seen in the dynamics simulations. The oxygen atom of the Ser in the peptide is about 4 Å from both D466 of PKCα and the γ-PO₄ group of ATP. It also was observed that the Na⁺ ions used to neutralize charges in the MD simulations were clustered around the PO₄ groups of the ATP. The position of these Na⁺ ions is similar to the Ca²⁺ ions in the crystal structure (PDB ID: 4XW5) of the catalytic conformation of PKA (Gerlits et al., supra). FIG. 10B is a snapshot with the closest distance of the poor activity peptide p12 to D466 and the APT γ-PO₄ group, extracted from the MD simulations. As anticipated, the catalytic distances in this low activity peptide were longer than that of high activity p2, leading to lower kinase activity of this peptide substrate. The MD ensemble for peptides p1, p2, p3, p9, p11, and p12 were clustered by the distances from the phosphorylated Ser/Thr to Asp466 and to the ATP γ-PO₄ group as shown in FIG. 10C. The population distribution showed that the highest population cluster for high activity peptides p1, p2, and p3 is around shorter distances and is much tighter as compared to the low activity counterparts p9, p11, and p12 (FIG. 10C). This suggested that peptides with high kinase activity are more likely than low activity peptides to populate catalytic conformations, as shown in FIG. 10D.

Crystal structures of PKA (Gerlits et al., J. Biol. Chem. 290:15538-15548, 2015) showed a rotamer flip in the χ₁ angle of the phosphorylated Ser/Thr, as shown in FIG. 10E. The side chain rotamer of the Ser/Thr in the peptide substrate flips its rotamer after PO₄ transfer to the Ser/Thr of the peptide. To determine whether the rotamer flip in the p-Ser/Thr of the substrate could be captured, MD simulations were performed after transferring the PO₄ group from ATP to the p-Ser/Thr in the substrate. To this end, the catalytic conformation with the shortest distance between Ser/Thr (OG) of peptide p1 and the γ-PO₄ of ATP, as well as to D466 (OD), was extracted as discussed in Example 7. The PO₄ group was then transferred to the Ser/Thr of the peptide, leaving ADP in the ATP binding site. All-atom MD simulations were performed on these two peptides bound to PKCα, and the rotamer conformations of the Ser/Thr residue in the peptide substrate were examined before and after phosphoryl transfer. FIG. 10F shows the rotamer flip of the χ₁ angle of the Ser/Thr in peptide substrates before and after the phosphoryl transfer. The rotamer change causes the Ser/Thr side chain to move away from Asp466 (FIG. 10F). The repulsion between the PO₄ group of the p-Ser/Thr and Asp466 may trigger the rotamer flip in the p-Ser/Thr, and this could be the initiating event for the peptide substrate to move away from the catalytic conformation before it dissociates from the kinase. In summary, these simulations showed that high activity peptides exhibit a higher population of the kinase-substrate conformations that are conducive to catalysis, with subsequent phosphoryl-transfer leading to a rotamer change for the p-Ser as seen in the crystal structures. The dynamics simulations described herein captured the conformational changes that lead to catalysis of the PO₄ transfer.

### Example 9—Basic Residues Two or Three Positions C-Terminal to the Phosphorylated Ser/Thr in the Peptide Substrate are Required for Good Kinase Activity

Studies were conducted to investigate why high activity peptides more frequently populate active conformations. Analysis of the MD simulation results showed that long range Coulombic attraction of the Arg/Lys two or three residues C-terminal to the phosphorylated residue (positions 11 and 12) to the γ-PO₄ group of the ATP ushers the Ser/Thr that is adjacent to this residue closer to the ATP, thereby priming phosphoryl-transfer. FIG. 11A shows a plot of the distance between the γ-PO₄ group of the ATP and the residues at position 9 (Ser) and 11 in peptide substrates p2 and p12 over time (in nanoseconds). For high activity peptide p2, Argil becomes close to the ATP γ-PO₄ group, thereby ushering in its neighbor Ser at position 9 toward the ATP. Such a long range Coulombic attraction between F11 in peptide p12 and the γ-PO₄ group of ATP is not possible in low activity peptide p12. FIG. 11B shows a snapshot from the MD simulations, demonstrating the proximity of the ATP γ-PO₄ group to S9 and R11 of peptide p2. To test this further using kinase Glo assays, these positions in low activity peptides p9, p10, and p12 were mutated to Arg/Lys (FIG. 4B). Mutation of residues at positions 11 and 12 in low activity peptides p9, p10, and p12 to Arg or Lys resulted in improved activity for all three peptides (FIG. 11C). Interestingly, peptide p2 has only two basic residues (R4 and R11), but it exhibits high kinase activity. Mutagenesis of R11 to Ile was sufficient to completely abolish the activity of this peptide (FIG. 11C). Adding an Arg substitution at position 12 (R11I plus G12R) retained partial activity of the peptide, attesting to the importance of the long-range Coulomb interaction mediated by an Arg/Lys residue 2 to 3 amino acids C-terminal to the phosphorylated Ser/Thr in driving kinase specific activity. These single and double point mutations to Arg in peptides p2, p9, p10, and p12 did not change the FRET ratios (and therefore the binding affinities) of the peptides, as shown in FIG. 11D.

### Example 10—High Activity Peptides Exhibit Greater Conformational Flexibility when Bound to the Kinase Catalytic Domain

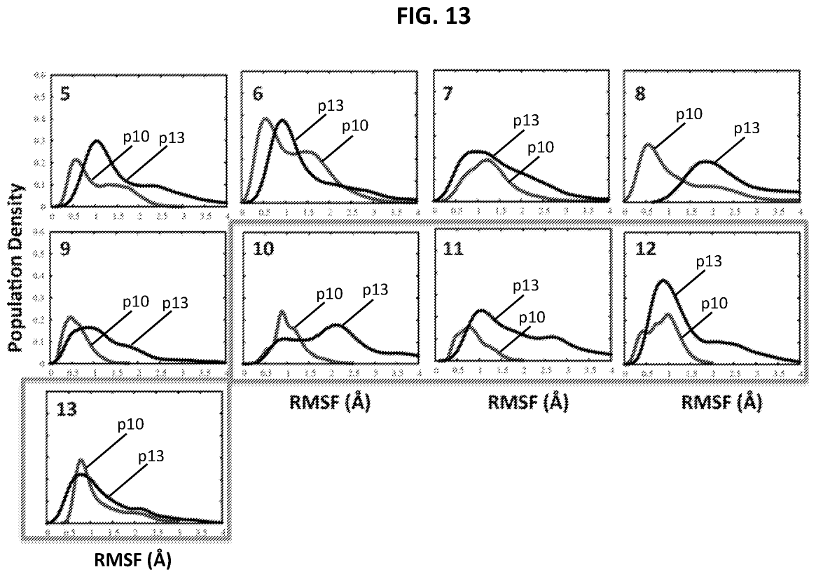

As seen in FIG. 4B, peptide substrates p10 and p13 showed similar binding affinities for PKCα, but p13 had a significantly different kinase activity. Studies were carried out to assess differences in the dynamics of p10 and p13 that may explain the differences in kinase activities. To examine the dynamics of the peptide substrate when bound to the kinase, the root mean square fluctuation (RMSF) of p10 was compared to that of p13. The RMSF for every residue in a peptide substrate reflects the flexibility of the peptide in the binding groove in PKCα. FIG. 13 shows the population density of RMSF for every residue in p10 and p13 when bound to PKCα during all-atom explicit MD simulations. The RMSF was calculated with respect to the average structure derived from the MD trajectories as reference. Position 9 is the position of the Ser/Thr that is phosphorylated by PKCα. While residues N-terminal to the phosphorylated Ser/Thr displayed overlapping distributions of RMSF, the C-terminal residues of p13 had a considerably broader distribution than p10. Peptide substrates p4, p5, and p13, which exhibit similar kinase activities, had substantial differences in binding affinity (FIG. 4B; p4>p5>p13). These three peptides showed similar flexibility (by RMSF) for all the residues in the peptides, but the interaction energies of the residues N-terminal to the p-Ser/Thr in these substrates corresponded to the reported binding strengths of these peptides (TABLE 3). Thus, higher flexibility in the region C-terminal to p-Ser/Thr in the peptide substrates corresponded to better kinase activity.

TABLE 3
Non-bonded interaction energies of residues in the
N-terminus of peptide substrates with the kinase.
STD is the standard deviation in the interaction energies.

| N-terminus | Int. Energy (-kcal/mol) | STD (-kcal/mol) |
|---|---|---|
| p4 | 129.4 | 15.5 |
| p5 | 100.0 | 11.8 |
| p13 | 225.9 | 21.3 |

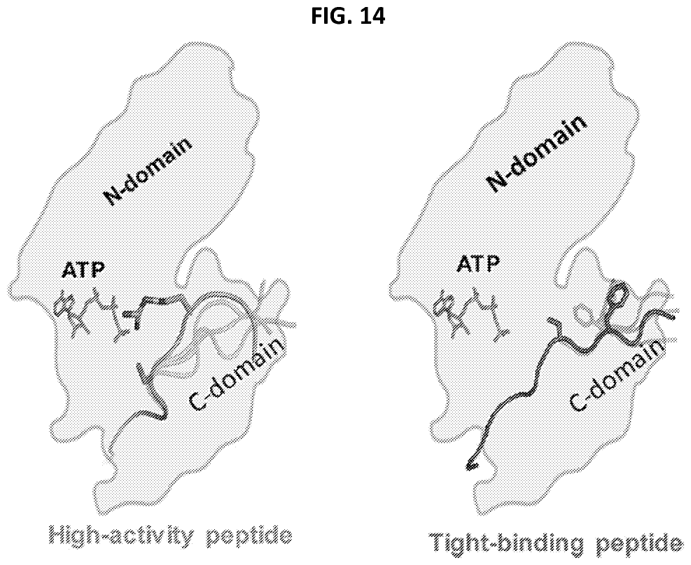

Taken together, these studies provided mechanistic insights into the structural and dynamic features of the PKCα-peptide interface that contribute to the binding and kinase activity of 14 different peptide substrates. Basic residues (Arg/Lys) located N-terminal to the pSer/Thr in the peptide substrates were found to contribute significantly to their binding affinity. The acidic residues Asp383, Asp470, Asp506, Glu533, Glu544, Glu548, and Asp542 in PKCα form a negatively charged patch to embed the Arg/Lys-rich N-terminus of tight binding peptide substrates. While this is in agreement with a metric for a consensus motif in the peptide sequences, as described elsewhere (Nishikawa et al., supra), the consensus motif metric did not correlate with the kinase activity toward these peptides. It was observed that flexibility of the amino acid residues located C-terminal to the pSer/Thr in the peptides regulate the kinase activity: the more flexible the C-terminus of the peptide, the better is its activity toward PKCα. Further the Arg/Lys positioned one or two residues from the pSer/Thr plays an important role in long-range attraction of ATP and threads the Ser/Thr close to both the γ-phosphate of ATP and the catalytic residue Asp466 in PKCα. These dynamics lead to formation of catalytically competent conformations. As shown in FIG. 14, peptide substrates that show high structural flexibility when bound to PKCα increase the proportion of conformations that position the Ser/Thr residue in proximity to both the γ-PO₄ and the aspartic acid (Asp466) that is important for proton abstraction. Thus, the findings described herein explain the wide range of kinase-specific activity observed for a range of PKCα peptide

substrates.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying a modulator of enzyme activity, comprising:
(a) providing a polypeptide sensor, the sensor comprising a catalytic domain of an enzyme, a linker comprising an amino acid sequence flanked by a FRET donor and a FRET acceptor, and a substrate for the enzyme, wherein the linker separates the catalytic domain from the substrate;
(b) measuring an initial FRET ratio for the sensor;
(c) contacting the sensor with a candidate molecule;
(d) measuring a test FRET ratio for the sensor after contacting step (c); and
(e) identifying the candidate molecule as a modulator of the enzyme activity when the test FRET ratio is increased or decreased by at least 10% as compared to the initial FRET ratio.

2. The method of claim 1, wherein the enzyme is a kinase.

3-6. (canceled)

7. The method of claim 1, wherein the linker comprises an ER/K sequence comprising a substantially repeating sequence of glutamic acid, arginine, and lysine residues, and is about 50 to about 250 amino acids in length.

8. (canceled)

9. The method of claim 1, wherein the linker comprises the amino acid sequence set forth in SEQ ID NO:32, or comprises a sequence that is at least 95% identical to the sequence set forth in SEQ ID NO:32.

10. The polypeptide of claim 1, wherein the linker further comprises an N-terminal tobacco etch virus (TEV) protease cleavage site between the ER/K sequence and the FRET donor or acceptor that is closest to the peptide sequence, and wherein the polypeptide further comprises a (Gly-Ser-Gly)₂₋₄ amino acid sequence on either side of the FRET acceptor and the FRET donor, and between the TEV protease cleavage site and the ER/K sequence.

11-13. (canceled)

14. The method of claim 1, wherein the enzyme is PCKα, and wherein the substrate is a peptide that is 5 to 20 amino acids in length.

15. The method of claim 14, wherein the peptide comprises the sequence set forth in SEQ ID NO:29.

16. The method of claim 1, further comprising providing two or more sensors, each comprising a different substrate for the enzyme, measuring initial and test FRET ratios for each substrate, and identifying the candidate compound as a substrate-specific modulator of the enzyme activity when at least some of the test FRET ratios are not increased or decreased by at least 10% as compared to the initial FRET ratios.

17. A method for identifying a modulator of enzyme activity, comprising:
(a) providing a polypeptide sensor, the sensor comprising a catalytic domain of an enzyme, a linker comprising an amino acid sequence flanked by a FRET donor and a FRET acceptor, and a candidate modulator for the enzyme, wherein the linker separates the catalytic domain from the candidate modulator;
(b) measuring an initial FRET ratio for the sensor;
(c) contacting the sensor with a substrate for the enzyme;
(d) measuring a test FRET ratio for the sensor; and
(e) identifying the candidate as a modulator of the enzyme when the test FRET ratio is within about 10% of the initial FRET ratio.

18. The method of claim 17, further comprising contacting a plurality of samples of the sensor with different substrates for the enzyme, measuring initial and test FRET ratios for each substrate, and identifying the candidate modulator as a substrate-specific modulator of the enzyme when at least some of the test FRET ratios are increased or decreased by at least 10% as compared to the initial FRET ratios.

19. A polypeptide comprising:
a catalytic domain of an enzyme;
a linker comprising an amino acid sequence flanked by a Förster resonance energy transfer (FRET) donor and a FRET acceptor; and
a substrate having affinity for the catalytic domain of the enzyme,
wherein the linker separates the catalytic domain from the substrate.

20. The polypeptide of claim 19, wherein the enzyme is a kinase.

21-24. (canceled)

25. The polypeptide of claim 19, wherein the linker comprises an ER/K sequence comprising a substantially repeating sequence of glutamic acid, arginine, and lysine residues, and is about 50 to about 250 amino acids in length.

26. (canceled)

27. The polypeptide of claim 19, wherein the linker comprises the amino acid sequence set forth in SEQ ID NO:32, or comprises a sequence that is at least 95% identical to the sequence set forth in SEQ ID NO:32.

28. The polypeptide of claim 19, wherein the linker further comprises an N-terminal TEV protease cleavage site between the ER/K sequence and the FRET donor or acceptor that is closest to the peptide sequence, and wherein the polypeptide further comprises (Gly-Ser-Gly)₂₋₄ amino acid sequence on either side of the FRET acceptor and the FRET donor, and between the TEV protease cleavage site and the ER/K sequence.

29-31. (canceled)

32. The polypeptide of claim 19, wherein the enzyme is PCKα, and wherein the substrate is a peptide that is 5 to 20 amino acids in length.

33. The polypeptide of claim 32, wherein the peptide comprises the sequence set forth in SEQ ID NO:29.

34. The polypeptide of claim 19 comprising, in order from amino terminus to carboxy terminus:
a molecule having affinity for a catalytic domain of an enzyme;
a (Gly-Ser-Gly)₂₋₄ amino acid sequence;
a resonance energy transfer donor or acceptor;
a (Gly-Ser-Gly)₂₋₄ amino acid sequence;
a TEV protease cleavage site;
a (Gly-Ser-Gly)₂₋₄ amino acid sequence;
a linker comprising the amino acid sequence set forth in SEQ ID NO:32, or a sequence at least 95% identical to the sequence set forth in SEQ ID NO:32;
a (Gly-Ser-Gly)₂₋₄ amino acid sequence;
a partner for the resonance energy transfer donor or acceptor;
a (Gly-Ser-Gly)₂₋₄ amino acid sequence; and
the catalytic domain of the enzyme, or a portion of the catalytic domain for which the molecule has affinity.

35-36. (canceled)

37. A nucleic acid encoding the polypeptide of claim 19.

38. A host cell comprising the nucleic acid of claim 37.
